(19) United States
(12) Patent Application Publication
(10) Pub. No.: US 2026/0274886 A1
(43) Pub. Date: Sep. 17, 2026

(54) CRYSTALLINE FORMS OF A FARNESOID X RECEPTOR AGONIST

(71) Applicant: ALFASIGMA S.P.A., Bologna (IT)

(72) Inventor: Gabriel M. GALVIN, San Diego, CA (US)

(73) Assignee: ALFASIGMA S.P.A., Bologna (IT)

(21) Appl. No.: 19/128,892

(22) PCT Filed: Nov. 9, 2023
(86) PCT No.: PCT/US2023/079257

§ 371 Date: May 9, 2025

(51) Int. Cl. C07J 9/00

(52) U.S. Cl. CPC C07J 9/005

(57) ABSTRACT

Provided are crystalline forms of a farnesoid x receptor agonist, compositions comprising the same, and methods of making and using such crystalline forms.

### CROSS REFERENCE TO RELATED APPLICATIONS

This application is a U.S. National Stage Application filed under 35 U.S.C. § 371 of International Application No. PCT/US2023/079257 filed Nov. 9, 2023, which claims priority to U.S. Provisional Application No. 63/383,479 filed Nov. 11, 2022, the disclosure of each which is incorporated herein by reference in its entirety.

### FIELD

Provided are crystalline forms of 3α,7α,11β-trihydroxy-6α-ethyl-5β-cholan-24-oic acid, compositions comprising the same, and methods of making and using such crystalline forms.

### BACKGROUND

3α,7α, 11β-trihydroxy-6α-ethyl-5β-cholan-24-oic acid is disclosed in U.S. Pat. No. 9,611,289. The compound may be used for the treatment of FXR-mediated conditions, for example, liver diseases and conditions.

Active pharmaceutical ingredients can exist in different physical forms (e.g., liquid or solid in different crystalline, amorphous, hydrate, or solvate forms), which can vary the processability, stability, solubility, bioavailability, or pharmacokinetics (absorption, distribution, metabolism, excretion, or the like) and/or bioequivalency of the active pharmaceutical ingredient and pharmaceutical compositions comprising it.

In addition, isolation of a crystalline solid form may have advantages over an amorphous solid in terms of purification, stability, and solid handling. Discovery of a crystalline solid may provide for more control over purity of intermediates or final products than an amorphous solid during manufacturing or for different physical properties, such as solubility, dissolution, tableting, etc. if used as an active pharmaceutical ingredient.

Thus, there is a need to identify compounds having an advantageous solid form.

### SUMMARY

3α,7α,11β-trihydroxy-6α-ethyl-5β-cholan-24-oic acid is shown as Compound 1 below:

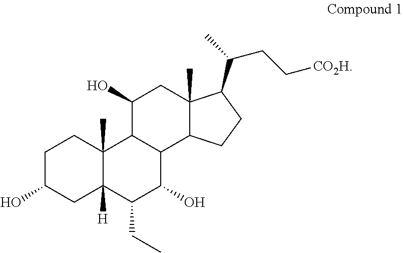 [SMILES: CC[C@H]1[C@@H](O)C2C3CC[C@H]([C@H](C)CCC(=O)O)[C@@]3(C)C[C@H](O)C2[C@@]2(C)CC[C@@H](O)C[C@@H]12]

Provided herein is Compound 1 (3α,7α,11β-trihydroxy-6α-ethyl-5β-cholan-24-oic acid) in a crystalline form. Crystalline forms of Compound 1 may be used as active ingredients in pharmaceutical compositions or as intermediates in synthetic processes described in U.S. Pat. Nos. 9,611,289 and 11,066,437, U.S. Publications Nos. 2018/0256600 and 2020/0164005, and International Publication No. WO 2018/226724, each of which is hereby incorporated by reference in its entirety.

### BRIEF DESCRIPTION OF THE DRAWINGS

FIG. 1 depicts an XRPD pattern of Crystalline Form 1 collected with Cu K radiation.

FIG. 2 depicts an overlay of a thermogravimetric analysis (TGA) thermogram and a differential analysis thermogram of Crystalline Form 1.

FIG. 3 depicts a differential scanning calorimetry thermogram of Crystalline Form 1.

FIG. 4 depicts a dynamic vapour sorption analysis of Crystalline Form 1.

FIG. 5 depicts an XRPD pattern of Crystalline Form 2 collected with Cu K radiation.

Vertical axis is Counts and horizontal axis is Position (°2θ) (Copper (Cu)).

FIG. 6 depicts an overlay of a thermogravimetric analysis (TGA) thermogram and a differential analysis thermogram of Crystalline Form 2.

FIG. 7 depicts an XRPD pattern of Crystalline Form 3 collected with Cu K radiation.

Vertical axis is Counts and horizontal axis is Position (°2θ) (Copper (Cu)).

FIG. 8 depicts an XRPD pattern of Crystalline Form 4 collected with Cu K radiation.

Vertical axis is Counts and horizontal axis is Position (°2θ) (Copper (Cu)).

FIG. 9 depicts an overlay of a thermogravimetric analysis (TGA) thermogram and a differential analysis thermogram of Crystalline Form 4.

FIG. 10 depicts an XRPD pattern of amorphous 3α,7α,11β-trihydroxy-6α-ethyl-5β-cholan-24-oic acid.

FIG. 11 depicts a chromatogram of amorphous 3α,7α,11β-trihydroxy-6α-ethyl-5β-cholan-24-oic acid (starting material) from Example 10.

FIG. 12 depicts a chromatogram of 3α,7α,11β-trihydroxy-6α-ethyl-5β-cholan-24-oic acid crystals from Example 10.

FIG. 13 depicts a chromatogram of 3α,7α,11β-trihydroxy-6α-ethyl-5β-cholan-24-oic acid (final product) from Example 10.

### DETAILED DESCRIPTION

Provided herein is Compound 1 (3α,7α,11β-trihydroxy-6α-ethyl-5β-cholan-24-oic acid) in crystalline form (e.g., Crystalline Form 1, Crystalline Form 2, Crystalline Form 3, or Crystalline Form 4 as described herein). The crystalline forms may be used in the pharmaceutical compositions, methods, kits, and syntheses described in U.S. Pat. Nos. 9,611,289 and 11,066,437, U.S. Publications Nos. 2018/0256600 and 2020/0164005, and International Publication No. WO 2018/226724, each of which is hereby incorporated by reference in its entirety.

The crystalline forms of Compound 1 described herein can be isolated and used as active pharmaceutical ingredients or as critical intermediates in the synthesis of Compound 1 for purging impurities, in particular on larger scale. Crystalline forms can be isolated in high purity from solvents with reduced toxicity (e.g., ICH solvents class 2 or class 3), substantially free of amorphous form. For instance, Crystalline Form 1 can be isolated from ICH class 3 solvents (e.g., alkane and/or ether solvents, e.g., tert-butyl methyl ether, heptane, and mixtures thereof) and is stable under stressed conditions (see, e.g., DVS and storage data in Example 6).

Accordingly, one of the embodiments of the present disclosure provides a Crystalline Form 1 (or Form 1) of Compound 1:

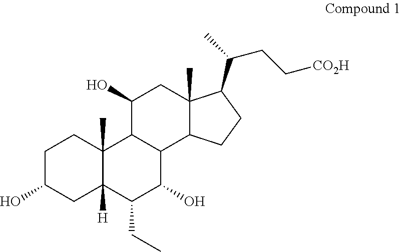 [SMILES: CC[C@H]1[C@@H](O)C2C3CC[C@H]([C@H](C)CCC(=O)O)[C@@]3(C)C[C@H](O)C2[C@@]2(C)CC[C@@H](O)C[C@@H]12]

Further provided are the following embodiments:
1.0) In one of the embodiments, provided is a Crystalline Form 1 (or Form 1) of Compound 1, wherein Form 1 exhibits an X-ray powder diffraction (XRPD) pattern comprising at least three, e.g., at least five, e.g., at least ten, of the following 2-theta (°) values: 5.4, 7.8, 10.9, 11.0, 12.4, 14.4, 15.5, 16.4, 16.7, 17.3, 18.3, and 19.7, wherein the XRPD is obtained using Cu K radiation.
1.1) Embodiment 1.0, wherein Form 1 exhibits an XRPD pattern comprising 2-theta (°) values of 5.4, 7.8, 10.9, 11.0, 12.4, 14.4, 15.5, 16.4, 16.7, 17.3, 18.3, and 19.7, wherein the XRPD is obtained using Cu K radiation.
1.2) Embodiment 1.0 or 1.1, wherein Form 1 exhibits an XRPD pattern comprising at least three, e.g., at least five, e.g., at least ten, e.g., at least twelve, of the following 2-theta (°) values: 5.4, 7.8, 9.4, 10.9, 11.0, 12.4, 14.4, 15.5, 16.4, 16.7, 17.3, 18.3, 19.2, 19.7, 20.0, 20.5, 22.0, 22.7, 23.0, 23.5, 24.3, 24.6, 25.0, 25.7, 26.4, 27.7, 28.6, 29.7, 30.3, 30.7, 31.2, 31.7, 32.7, and 34.1, wherein the XRPD is obtained using Cu K radiation.
1.3) Any of Embodiments 1.0, 1.1, or 1.2, wherein Form 1 exhibits an XRPD pattern comprising 2-theta (°) values of 5.4, 7.8, 9.4, 10.9, 11.0, 12.4, 14.4, 15.5, 16.4, 16.7, 17.3, 18.3, 19.2, 19.7, 20.0, 20.5, 22.0, 22.7, 23.0, 23.5, 24.3, 24.6, 25.0, 25.7, 26.4, 27.7, 28.6, 29.7, 30.3, 30.7, 31.2, 31.7, 32.7, and 34.1, wherein the XRPD is obtained using Cu K radiation.
1.4) Any of Embodiments 1.0 et seq., wherein Form 1 exhibits an XRPD pattern comprising at least three, e.g., at least five, e.g., at least ten, of the following 2-theta (°) values: 5.45, 7.79, 10.89, 10.95, 12.40, 14.43, 15.47, 16.38, 16.74, 17.32, 18.26, and 19.75, wherein the XRPD is obtained using Cu K radiation.
1.5) Any of Embodiments 1.0 et seq., wherein Form 1 exhibits an XRPD pattern comprising 2-theta (°) values of 5.45, 7.79, 10.89, 10.95, 12.40, 14.43, 15.47, 16.38, 16.74, 17.32, 18.26, and 19.75, wherein the XRPD is obtained using Cu K radiation.
1.6) Any of Embodiments 1.0 et seq., wherein Form 1 exhibits an XRPD pattern comprising at least three, e.g., at least five, e.g., at least ten, e.g., at least twelve, of the following 2-theta (°) values: 5.45, 7.79, 9.44, 10.89, 10.95, 12.40, 14.43, 15.47, 16.38, 16.74, 17.32, 18.26, 19.25, 19.75, 20.03, 20.49, 21.99, 22.65, 23.04, 23.49, 24.32, 24.58, 24.96, 25.75, 26.40, 27.65, 28.63, 29.68, 30.32, 30.72, 31.22, 31.70, 32.69, and 34.09, wherein the XRPD is obtained using Cu K radiation.
1.7) Any of Embodiments 1.0 et seq., wherein Form 1 exhibits exhibiting an XRPD pattern comprising 2-theta (°) values of 5.45, 7.79, 9.44, 10.89, 10.95, 12.40, 14.43, 15.47, 16.38, 16.74, 17.32, 18.26, 19.25, 19.75, 20.03, 20.49, 21.99, 22.65, 23.04, 23.49, 24.32, 24.58, 24.96, 25.75, 26.40, 27.65, 28.63, 29.68, 30.32, 30.72, 31.22, 31.70, 32.69, and 34.09, wherein the XRPD is obtained using Cu K radiation.
1.8) Any of Embodiments 1.0 et seq., wherein Form 1 exhibits an XRPD pattern comprising at least three, e.g., at least five, e.g., at least ten, e.g., at least twelve, of the 2-theta (°) values set forth in Table A below:

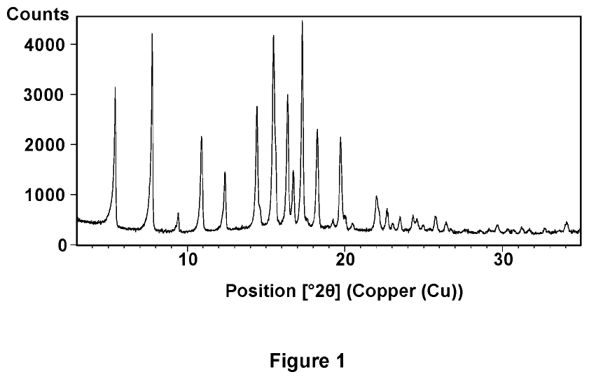

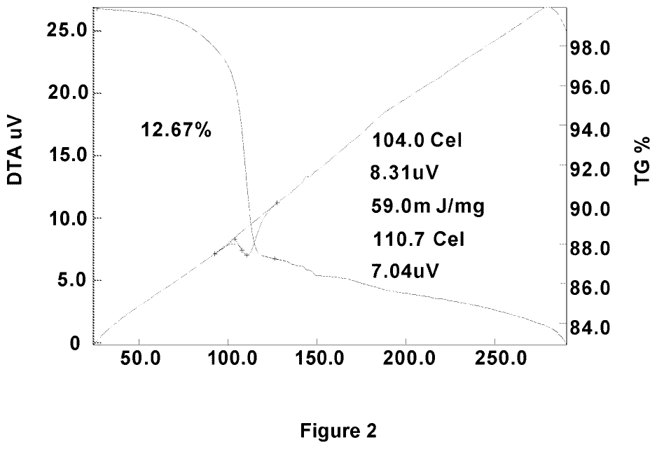

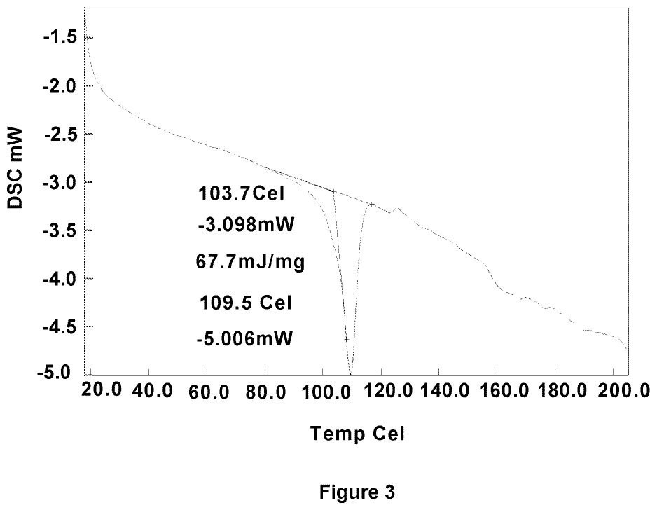

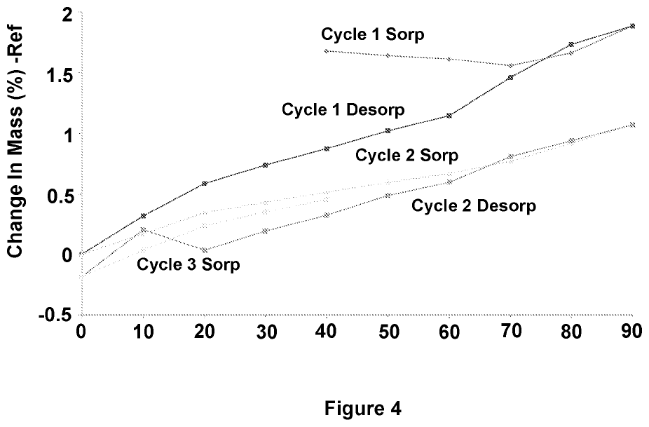

TABLE A

| | Pos. | d-spacing | Rel. Int. |
|---|---|---|---|
| No. | [°2θ] | [Å] | [%] |
| 1 | 5.446 | 16.229 | 67.23 |
| 2 | 7.787 | 11.354 | 93.32 |
| 3 | 9.439 | 9.370 | 9.00 |
| 4 | 10.891 | 8.124 | 45.65 |
| 5 | 10.953 | 8.078 | 39.41 |
| 6 | 12.401 | 7.138 | 28.29 |
| 7 | 14.431 | 6.138 | 61.11 |
| 8 | 15.467 | 5.729 | 94.71 |
| 9 | 16.380 | 5.412 | 64.17 |
| 10 | 16.740 | 5.296 | 29.36 |
| 11 | 17.316 | 5.121 | 100.00 |
| 12 | 18.257 | 4.859 | 49.84 |
| 13 | 19.247 | 4.612 | 5.82 |
| 14 | 19.745 | 4.496 | 45.05 |
| 15 | 20.034 | 4.432 | 7.19 |
| 16 | 20.490 | 4.335 | 4.11 |
| 17 | 21.987 | 4.043 | 17.57 |
| 18 | 22.651 | 3.926 | 10.19 |
| 19 | 23.036 | 3.861 | 4.11 |
| 20 | 23.494 | 3.787 | 7.32 |
| 21 | 24.319 | 3.660 | 7.79 |
| 22 | 24.582 | 3.621 | 6.30 |
| 23 | 24.957 | 3.568 | 3.38 |
| 24 | 25.748 | 3.460 | 7.48 |
| 25 | 26.395 | 3.377 | 4.34 |
| 26 | 27.650 | 3.226 | 1.05 |
| 27 | 28.631 | 3.118 | 0.73 |
| 28 | 29.681 | 3.010 | 3.76 |
| 29 | 30.319 | 2.948 | 1.39 |
| 30 | 30.717 | 2.911 | 1.10 |
| 31 | 31.221 | 2.865 | 2.10 |
| 32 | 31.699 | 2.823 | 1.28 |
| 33 | 32.691 | 2.739 | 1.98 |
| 34 | 34.093 | 2.630 | 4.89, |

wherein the XRPD is obtained using Cu K radiation.
1.9) Any of Embodiments 1.0 et seq., wherein Form 1 exhibits an XRPD pattern comprising the 2-theta (°) values set forth in Table A of Embodiment 1.8, wherein the XRPD is obtained using Cu K radiation.
1.10) Any of Embodiments 1.0 et seq., wherein Form 1 exhibits an XRPD pattern comprising at least three, e.g., at least five, e.g., at least ten, of the following d-spacing (Å) values: 16.2, 11.4, 8.1, 7.1, 6.1, 5.7, 5.4, 5.3, 5.1, 4.9, and 4.5.
1.11) Any of Embodiments 1.0 et seq., wherein Form 1 exhibits an XRPD pattern comprising d-spacing (Å) values of 16.2, 11.4, 8.1, 7.1, 6.1, 5.7, 5.4, 5.3, 5.1, 4.9, and 4.5.
1.12) Any of Embodiments 1.0 et seq., wherein Form 1 exhibits an XRPD pattern comprising at least three, e.g., at least five, e.g., at least ten, e.g., at least twelve, of the following d-spacing (Å) values: 16.2, 11.4, 9.4, 8.1, 7.1, 6.1, 5.7, 5.4, 5.3, 5.1, 4.9, 4.6, 4.5, 4.4, 4.3, 4.0, 3.9, 3.8, 3.7, 3.6, 3.5, 3.4, 3.2, 3.1, 3.0, 2.9, 2.8, 2.7, and 2.6.
1.13) Any of Embodiments 1.0 et seq., wherein Form 1 exhibits an XRPD pattern comprising d-spacing (Å) values of 16.2, 11.4, 9.4, 8.1, 7.1, 6.1, 5.7, 5.4, 5.3, 5.1, 4.9, 4.6, 4.5, 4.4, 4.3, 4.0, 3.9, 3.8, 3.7, 3.6, 3.5, 3.4, 3.2, 3.1, 3.0, 2.9, 2.8, 2.7, and 2.6.
1.14) Any of Embodiments 1.0 et seq., wherein Form 1 exhibits an XRPD pattern comprising at least three, e.g., at least five, e.g., at least ten, of the following d-spacing (Å) values: 16.23, 11.35, 8.12, 8.08, 7.14, 6.14, 5.73, 5.41, 5.30, 5.12, 4.86, and 4.50.
1.15) Any of Embodiments 1.0 et seq., wherein Form 1 exhibits an XRPD pattern comprising d-spacing (Å) values of 16.23, 11.35, 8.12, 8.08, 7.14, 6.14, 5.73, 5.41, 5.30, 5.12, 4.86, and 4.50.
1.16) Any of Embodiments 1.0 et seq., wherein Form 1 exhibits an XRPD pattern comprising at least three, e.g., at least five, e.g., at least ten, e.g., at least twelve, of the following d-spacing (Å) values: 16.23, 11.35, 9.37, 8.12, 8.08, 7.14, 6.14, 5.73, 5.41, 5.30, 5.12, 4.86, 4.61, 4.50, 4.43, 4.34, 4.04, 3.93, 3.86, 3.79, 3.66, 3.62, 3.57, 3.46, 3.38, 3.23, 3.12, 3.01, 2.95, 2.91, 2.87, 2.82, 2.74, and 2.63.
1.17) Any of Embodiments 1.0 et seq., wherein Form 1 exhibits an XRPD pattern comprising d-spacing (Å) values of 16.23, 11.35, 9.37, 8.12, 8.08, 7.14, 6.14, 5.73, 5.41, 5.30, 5.12, 4.86, 4.61, 4.50, 4.43, 4.34, 4.04, 3.93, 3.86, 3.79, 3.66, 3.62, 3.57, 3.46, 3.38, 3.23, 3.12, 3.01, 2.95, 2.91, 2.87, 2.82, 2.74, and 2.63.
1.18) Any of Embodiments 1.0 et seq., wherein Form 1 exhibits an XRPD pattern comprising at least three, e.g., at least five, e.g., at least ten, e.g., at least twelve, of the d-spacing (Å) values set forth in Table A of Embodiment 1.8.
1.19) Any of Embodiments 1.0 et seq., wherein Form 1 exhibits an XRPD pattern comprising the d-spacing (Å) values set forth in Table A of Embodiment 1.8.
1.20) Any of Embodiments 1.0 et seq., wherein Form 1 exhibits an XRPD pattern comprising at least three, e.g., at least five, e.g., at least ten, e.g., at least twelve, e.g., all of the peaks, of the XRPD shown in FIG. 1, wherein the XRPD is obtained using Cu K radiation.
1.21) Any of Embodiments 1.0 et seq., wherein Form 1 exhibits an XRPD pattern comprising characteristic peaks of the XRPD pattern shown in FIG. 1, wherein the XRPD is obtained using Cu K radiation.
1.22) Any of Embodiments 1.0 et seq., wherein Form 1 exhibits an XRPD pattern comprising representative peaks of the XRPD pattern shown in FIG. 1, wherein the XRPD is obtained using Cu K radiation.
1.23) Any of Embodiments 1.0 et seq., wherein Form 1 exhibits an XRPD pattern corresponding to FIG. 1, wherein the XRPD is obtained using Cu K radiation.
1.24) Any of Embodiments 1.0 et seq., wherein Form 1 exhibits a thermogravimetric analysis (TGA) thermogram comprising weight loss between 20° C. and 120° C., e.g., a weight loss of 11-14 weight %, e.g., a weight loss of 12-13 weight %, e.g., a weight loss of 12.7 weight %. For instance, any of Embodiments 1.0 et seq., wherein Form 1 exhibits a thermogravimetric analysis (TGA) thermogram corresponding to FIG. 2.
1.25) Any of Embodiments 1.0 et seq., wherein Form 1 exhibits differential thermal analysis thermogram comprising an endothermic event, e.g., an endotherm at 111° C., e.g., an endotherm at 111° C. with an onset at 104° C. For instance, any of Embodiments 1.0 et seq., wherein Form 1 exhibits a differential analysis thermogram corresponding to FIG. 2.
1.26) Any of Embodiments 1.0 et seq., wherein Form 1 exhibits a differential scanning calorimetry comprising an endotherm, e.g., an endotherm at 110° C., e.g., an endotherm at 110° C. with an onset at 104° C. For instance, any of Embodiments 1.0 et seq., wherein Form 1 exhibits a differential scanning calorimetry thermogram corresponding to FIG. 3.
1.27) Any of Embodiments 1.0 et seq., wherein Form 1 exhibits a dynamic vapour sorption analysis comprising 3% or less weight gain upon increasing relative humidity from 0-90%, e.g., 2% or less weight gain, e.g., 1.5% or less weight gain.
1.28) Any of Embodiments 1.0 et seq., wherein Form 1 exhibits a dynamic vapour sorption analysis comprising 3% or less weight loss upon increasing relative humidity from 0-90%, e.g., 2% or less weight loss.
1.29) Any of Embodiments 1.0 et seq., wherein Form 1 exhibits a dynamic vapour sorption analysis corresponding to FIG. 4.
1.30) Any of Embodiments 1.0 et seq., wherein Karl Fischer analysis shows a moisture content of <1% w/w, e.g., 0.6% w/w.
1.31) Any of Embodiments 1.0 et seq., wherein Form 1 comprises 0.7 equivalents tBME, e.g., as measured by ¹H NMR. For instance, any of Embodiments 1.0 et seq., wherein Form 1 comprises 0.67 equivalents tBME, e.g., as measured by ¹H NMR.
1.32) Any of Embodiments 1.0 et seq., wherein preparation of Form 1 comprises mixing, optionally with stirring, shaking, heating, and/or cooling, 3α,7α,11β-trihydroxy-6α-ethyl-5β-cholan-24-oic acid with one or more solvents (e.g., an ICH class 2 or 3 solvent). The one or more solvents may be ones in which 3α,7α,11β-trihydroxy-6α-ethyl-5β-cholan-24-oic acid is soluble (e.g., tert-buty methyl ether and/or methyl ethyl ketone) or less soluble (e.g., poorly soluble) (e.g., water, diisopropyl ether, or a liquid hydrocarbon, e.g., hexane, heptane, and/or toluene).
1.33) Any of Embodiments 1.0 et seq., wherein preparation of Form 1 comprises mixing, optionally with stirring, shaking, heating, and/or cooling, Compound 1 with a first solvent in which Compound 1 is soluble (e.g., tert-butyl methyl ether and/or methyl ethyl ketone) and a second solvent in which Compound 1 is poorly soluble (e.g., water, diisopropyl ether, or a liquid hydrocarbon, e.g., hexane, heptane, and/or toluene).
1.34) Embodiment 1.33, wherein volume ratio of the first solvent to the second solvent is 1:1 to 20:1, e.g., 1:1 to 10:1, e.g., 1:1 to 9:1 e.g., 1:1 to 5:1, e.g., 1:1 to 4:1, e.g., 1:1 to 2.3:1. For instance, Embodiment 1.33, wherein volume ratio of the first solvent to the second solvent is 4:1. Examples of solvents in which 3α,7α,11β-trihydroxy-6α-ethyl-5β-cholan-24-oic acid is soluble are tert-butyl methyl ether and methyl ethyl ketone. Examples of solvents in which 3α,7α,11β-trihydroxy-6α-ethyl-5β-cholan-24-oic acid is poorly soluble are hexane, heptane, toluene, and water. Or, Embodiment 1.33, wherein volume ratio of the first solvent to the second solvent is 1:20 to 1:1, e.g., 1:10 to 1:1, e.g., 1:4 to 1:1, e.g., 1:1.
1.35) Embodiment 1.33 or 1.34, wherein the first solvent is tert-butyl methyl ether or methyl ethyl ketone. For instance, Embodiment 1.33 or 1.34, wherein the first solvent is tert-butyl methyl ether.
1.36) Any of Embodiments 1.33-1.35, wherein the second solvent is hexane, heptane, toluene, or water. For instance, any of Embodiments 1.33-1.35, wherein the second solvent is heptane. Or, for instance, any of Embodiments 1.33-1.35, wherein the second solvent is water. Or, for instance, any of Embodiments 1.33-1.35, wherein the second solvent is toluene.
1.37) Any of Embodiments 1.0-1.32, wherein preparation of Form 1 comprises mixing, optionally with stirring, shaking, heating, and/or cooling, 3α,7α,11β-trihydroxy-6α-ethyl-5β-cholan-24-oic acid with one or more solvents in which 3α,7α,11β-trihydroxy-6α-ethyl-5β-cholan-24-oic acid is poorly soluble (e.g., water, diisopropyl ether, and/or a liquid hydrocarbon, e.g., hexane, heptane, and/or toluene). For instance, any of Embodiments 1.0-1.32, wherein preparation of Form 1 comprises mixing, optionally with stirring, shaking, heating, and/or cooling, 3α,7α,11 β-trihydroxy-6α-ethyl-5β-cholan-24-oic acid with two or more solvents in which 3α,7α,11β-trihydroxy-6α-ethyl-5β-cholan-24-oic acid is poorly soluble (e.g., water, diisopropyl ether, and/or a liquid hydrocarbon, e.g., hexane, heptane, and/or toluene).

For instance, any of Embodiments 1.0-1.32, wherein preparation of Form 1 comprises mixing, optionally with stirring, shaking, heating, and/or cooling, 3α,7α,11β-trihydroxy-6α-ethyl-5β-cholan-24-oic acid with two solvents in which 3α,7α,11β-trihydroxy-6α-ethyl-5β-cholan-24-oic acid is poorly soluble and the volume ratio of the liquids is 5:1 to 1:1, e.g., 1:1 (e.g., 1:1 diisopropyl ether:hexane or heptane).
1.38) Embodiment 1.37, wherein 3α,7α,11β-trihydroxy-6α-ethyl-5β-cholan-24-oic acid is mixed with diisopropyl ether and hexane or heptane.
1.39) Any of Embodiments 1.0 et seq., wherein preparation of Form 1 comprises mixing, optionally with stirring, shaking, heating, and/or cooling, 3α,7α,11β-trihydroxy-6α-ethyl-5β-cholan-24-oic acid with one or more of tert-butyl methyl ether, methyl ethyl ketone, diisopropyl ether, hexane, heptane, toluene, and water. For instance, any of Embodiments 1.0 et seq., wherein preparation of Form 1 comprises mixing, optionally with stirring, shaking, heating, and/or cooling, 3α,7α,11β-trihydroxy-6α-ethyl-5β-cholan-24-oic acid with tert-butyl methyl ether, diisopropyl ether, or methyl ethyl ketone and hexane or heptane. Or, for instance, any of Embodiments 1.0 et seq., wherein preparation of Form 1 comprises mixing, optionally with stirring, shaking, heating, and/or cooling, 3α,7α,11β-trihydroxy-6α-ethyl-5β-cholan-24-oic acid with tert-butyl methyl ether and water (e.g., tert-butyl methyl ether saturated water). Or, for instance, any of Embodiments 1.0 et seq., wherein preparation of Form 1 comprises mixing, optionally with stirring, shaking, heating, and/or cooling, 3α,7α,11β-trihydroxy-6α-ethyl-5β-cholan-24-oic acid with tert-butyl methyl ether and toluene.
1.40) Any of Embodiments 1.32-1.39, wherein the mixture of 3α,7α,11β-trihydroxy-6α-ethyl-5β-cholan-24-oic acid and the one or more solvents is heated, e.g., above room temperature to 60° C., e.g., between 40° C. to 50° C., e.g., to 40° C. or 50° C. For instance, any of Embodiments 1.32-1.39, wherein the mixture of 3α,7,11β-trihydroxy-6α-ethyl-5β-cholan-24-oic acid with tert-butyl methyl ether and liquid hydrocarbon (e.g., heptane) is heated, e.g., to 40° C. or 50° C.
1.41) Any of Embodiments 1.32-1.40, wherein the mixture of 3α,7α,11β-trihydroxy-6α-ethyl-5β-cholan-24-oic acid and the one or more solvents is thermally cycled, e.g., heated and cooled, e.g., between 50° C. and 0° C., e.g., between 40° C. and 5° C. For instance, any of Embodiments 1.32-1.40, wherein the mixture of 3α,7α,11β-trihydroxy-6α-ethyl-5β-cholan-24-oic acid with tert-butyl methyl ether and liquid hydrocarbon (e.g., heptane) is thermally cycled, e.g., heated and cooled, e.g., between 40° C. and 5° C.
1.42) Any of Embodiments 1.32-1.41 further comprising allowing the one or more solvents to evaporate, e.g., at ambient conditions or under vacuum.
1.43) Any of Embodiments 1.32-1.42, wherein the mixture is cooled below room temperature, e.g., between −20° C. to 10° C., e.g., between 10° C. and 0° C., e.g., between 5° C. and 0° C.
1.44) Any of Embodiments 1.0 et seq., wherein preparation of Form 1 comprises seeding with Crystalline Form 1 (e.g., any of Embodiments 1.0 et seq.).
1.45) Any of Embodiments 1.32-1.44 further comprising isolating Form 1.
1.46) Embodiment 1.45, wherein Form 1 is isolated by centrifugation and/or filtration.
1.47) Any of Embodiments 1.0 et seq., wherein Form 1 is made by any of Process 1 et seq., vide infra.
1.48) Any of Embodiments 1.0 et seq., wherein Form 1 is made as described in any of the examples that produce Form 1.
1.49) Any of Embodiments 1.0 et seq., wherein each of the 2-theta (°) values of the XRPD pattern have an acceptable deviation of ±0.2°.
1.50) Any of Embodiments 1.0 et seq., wherein Form 1 is a tert-butyl methyl ether solvate (e.g., a 1 equivalent of tert-butyl methyl ether solvate).

Another embodiment of the present disclosure provides a Crystalline Form 2 (or Form 2) of Compound 1:

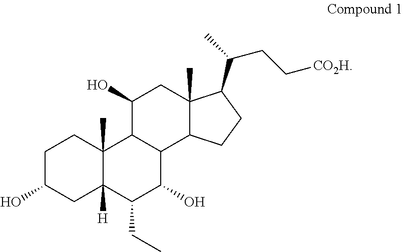 [SMILES: CC[C@H]1[C@@H](O)C2C3CC[C@H]([C@H](C)CCC(=O)O)[C@@]3(C)C[C@H](O)C2[C@@]2(C)CC[C@@H](O)C[C@@H]12]

Further provided are the following embodiments:
2.0) In one of the embodiments, provided is a Crystalline Form 2 (or Form 2) of Compound 1, wherein Form 2 exhibits an XRPD pattern comprising at least three, e.g., at least five, e.g., at least ten, e.g., at least fifteen, e.g., at least twenty, e.g., at least twenty-five, e.g., at least thirty, of the following 2-theta (°) values: 4.4, 5.5, 6.0, 7.3, 8.5, 8.9, 9.5, 10.3, 10.7, 11.4, 12.2, 12.6, 14.1, 14.8, 16.5, 17.1, 17.6, 18.1, 18.7, 19.1, 19.3, 19.5, 20.0, 20.5, 20.9, 21.2, 21.9, 22.6, 23.3, 24.3, and 25.2, wherein the XRPD is obtained using Cu K radiation.
2.1) Embodiment 2.0, wherein Form 2 exhibits an XRPD pattern comprising 2-theta (°) values of 4.4, 5.5, 6.0, 7.3, 8.5, 8.9, 9.5, 10.3, 10.7, 11.4, 12.2, 12.6, 14.1, 14.8, 16.5, 17.1, 17.6, 18.1, 18.7, 19.1, 19.3, 19.5, 20.0, 20.5, 20.9, 21.2, 21.9, 22.6, 23.3, 24.3, and 25.2, wherein the XRPD is obtained using Cu K radiation.
2.2) Embodiment 2.0 or 2.1, wherein Form 2 exhibits an XRPD pattern comprising at least three, e.g., at least five, e.g., at least ten, e.g., at least fifteen, e.g., at least twenty, e.g., at least twenty-five, e.g., at least thirty, e.g., at least thirty-one, of the following 2-theta (°) values: 4.4, 5.5, 6.0, 7.0, 7.3, 7.9, 8.5, 8.9, 9.5, 10.3, 10.7, 11.4, 11.7, 12.2, 12.6, 12.9, 13.7, 14.1, 14.8, 16.5, 17.1, 17.6, 18.1, 18.7, 19.1, 19.3, 19.5, 20.0, 20.5, 20.9, 21.2, 21.9, 22.6, 23.3, 24.3, 24.5, 25.2, 25.5, 25.9, and 29.1, wherein the XRPD is obtained using Cu K radiation.
2.3) Any of Embodiments 2.0, 2.1, or 2.2, wherein Form 2 exhibits an XRPD pattern comprising 2-theta (°) values of 4.4, 5.5, 6.0, 7.0, 7.3, 7.9, 8.5, 8.9, 9.5, 10.3, 10.7, 11.4, 11.7, 12.2, 12.6, 12.9, 13.7, 14.1, 14.8, 16.5, 17.1, 17.6, 18.1, 18.7, 19.1, 19.3, 19.5, 20.0, 20.5, 20.9, 21.2, 21.9, 22.6, 23.3, 24.3, 24.5, 25.2, 25.5, 25.9, and 29.1, wherein the XRPD is obtained using Cu K radiation.
2.4) Any of Embodiments 2.0 et seq., wherein Form 2 exhibits an XRPD pattern comprising at least three, e.g., at least five, e.g., at least ten, e.g., at least fifteen, e.g., at least twenty, e.g., at least twenty-five, e.g., at least thirty, of the following 2-theta (°) values: 4.45, 5.46, 6.02, 7.29, 8.51, 8.92, 9.46, 10.26, 10.74, 11.39, 12.24, 12.60, 14.13, 14.83, 16.53, 17.06, 17.62, 18.12, 18.66, 19.09, 19.27, 19.52, 20.04, 20.51, 20.87, 21.22, 21.91, 22.62, 23.33, 24.32, and 25.23, wherein the XRPD is obtained using Cu K radiation.
2.5) Any of Embodiments 2.0 et seq., wherein Form 2 exhibits an XRPD pattern comprising 2-theta (°) values of 4.45, 5.46, 6.02, 7.29, 8.51, 8.92, 9.46, 10.26, 10.74, 11.39, 12.24, 12.60, 14.13, 14.83, 16.53, 17.06, 17.62, 18.12, 18.66, 19.09, 19.27, 19.52, 20.04, 20.51, 20.87, 21.22, 21.91, 22.62, 23.33, 24.32, and 25.23, wherein the XRPD is obtained using Cu K radiation.
2.6) Any of Embodiments 2.0 et seq., wherein Form 2 exhibits an XRPD pattern comprising at least three, e.g., at least five, e.g., at least ten, e.g., at least fifteen, e.g., at least twenty, e.g., at least twenty-five, e.g., at least thirty, e.g., at least thirty-one, of the following 2-theta (°) values: 4.45, 5.46, 6.02, 7.04, 7.29, 7.94, 8.51, 8.92, 9.46, 10.26, 10.74, 11.39, 11.67, 12.24, 12.60, 12.93, 13.73, 14.13, 14.83, 16.53, 17.06, 17.62, 18.12, 18.66, 19.09, 19.27, 19.52, 20.04, 20.51, 20.87, 21.22, 21.91, 22.62, 23.33, 24.32, 24.54, 25.23, 25.54, 25.89, and 29.09, wherein the XRPD is obtained using Cu K radiation.
2.7) Any of Embodiments 2.0 et seq., wherein Form 2 exhibits an XRPD pattern comprising 2-theta (°) values of 4.45, 5.46, 6.02, 7.04, 7.29, 7.94, 8.51, 8.92, 9.46, 10.26, 10.74, 11.39, 11.67, 12.24, 12.60, 12.93, 13.73, 14.13, 14.83, 16.53, 17.06, 17.62, 18.12, 18.66, 19.09, 19.27, 19.52, 20.04, 20.51, 20.87, 21.22, 21.91, 22.62, 23.33, 24.32, 24.54, 25.23, 25.54, 25.89, and 29.09, wherein the XRPD is obtained using Cu K radiation.
2.8) Any of Embodiments 2.0 et seq., wherein Form 2 exhibits an XRPD pattern comprising at least three, e.g., at least five, e.g., at least ten, e.g., at least fifteen, e.g., at least twenty, e.g., at least twenty-five, e.g., at least thirty, e.g., at least thirty-one, of the 2-theta (°) values set forth in Table B below:

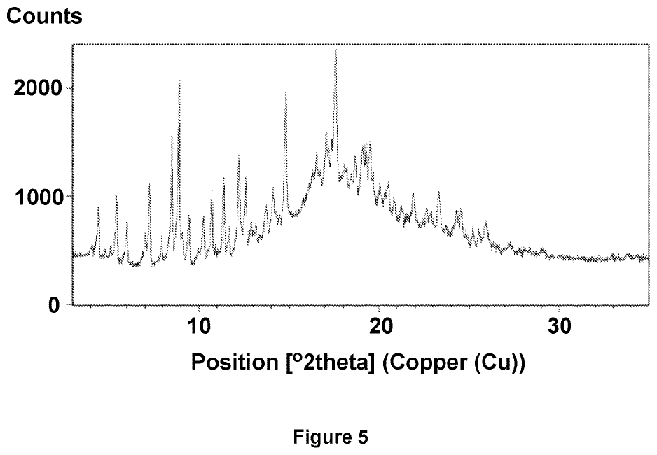

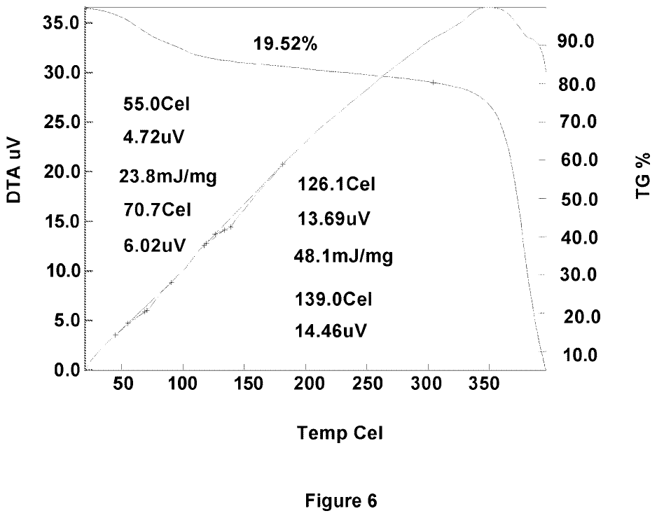

TABLE B

| | Pos. | d-spacing | Rel. Int. |
|---|---|---|---|
| No. | [°2θ] | [Å] | [%] |
| 1 | 4.447 | 19.870 | 26.62 |
| 2 | 5.463 | 16.177 | 36.26 |
| 3 | 6.022 | 14.678 | 23.72 |
| 4 | 7.037 | 12.562 | 18.72 |
| 5 | 7.287 | 12.131 | 43.30 |
| 6 | 7.940 | 11.135 | 15.90 |
| 7 | 8.512 | 10.389 | 69.19 |
| 8 | 8.916 | 9.918 | 100.00 |
| 9 | 9.460 | 9.350 | 25.41 |
| 10 | 10.256 | 8.625 | 23.15 |
| 11 | 10.741 | 8.237 | 34.78 |
| 12 | 11.388 | 7.770 | 39.68 |
| 13 | 11.674 | 7.581 | 12.17 |
| 14 | 12.239 | 7.232 | 45.85 |
| 15 | 12.598 | 7.026 | 36.75 |
| 16 | 12.927 | 6.848 | 10.69 |
| 17 | 13.731 | 6.449 | 15.89 |
| 18 | 14.132 | 6.267 | 23.84 |
| 19 | 14.833 | 5.973 | 69.38 |
| 20 | 16.532 | 5.363 | 36.32 |
| 21 | 17.062 | 5.197 | 47.48 |
| 20 | 17.062 | 5.197 | 52.28 |
| 21 | 17.624 | 5.032 | 63.39 |
| 22 | 18.116 | 4.897 | 100.00 |
| 23 | 18.660 | 4.755 | 44.00 |
| 24 | 19.090 | 4.649 | 49.61 |
| 25 | 19.272 | 4.606 | 55.20 |
| 26 | 19.521 | 4.548 | 54.78 |
| 27 | 20.036 | 4.432 | 55.68 |
| 28 | 20.510 | 4.330 | 35.27 |
| 29 | 20.865 | 4.257 | 35.58 |
| 30 | 21.221 | 4.187 | 26.86 |
| 31 | 21.905 | 4.058 | 22.99 |
| 32 | 22.617 | 3.931 | 29.53 |
| 33 | 23.332 | 3.813 | 21.43 |
| 34 | 24.318 | 3.660 | 29.59 |
| 35 | 24.540 | 3.628 | 19.64 |
| 36 | 25.227 | 3.530 | 21.16 |
| 37 | 25.544 | 3.487 | 9.08 |
| 38 | 25.890 | 3.441 | 9.47 |
| 39 | 29.087 | 3.070 | 12.82 |
| 40 | 29.087 | 3.070 | 2.47, |

wherein the XRPD is obtained using Cu K radiation.
2.9) Any of Embodiments 2.0 et seq., wherein Form 2 exhibits an XRPD pattern comprising the 2-theta (°) values set forth in Table B of Embodiment 2.8, wherein the XRPD is obtained using Cu K radiation.
2.10) Any of Embodiments 2.0 et seq., wherein Form 2 exhibits an XRPD pattern comprising at least three, e.g., at least five, e.g., at least ten, e.g., at least fifteen, e.g., at least twenty, e.g., at least twenty-five, of the following d-spacing (Å) values: 19.9, 16.2, 14.7, 12.1, 10.4, 9.9, 9.4, 8.6, 8.2, 7.8, 7.2, 7.0, 6.3, 6.0, 5.4, 5.2, 5.0, 4.9, 4.8, 4.6, 4.5, 4.4, 4.3, 4.2, 4.1, 3.9, 3.8, 3.7, and 3.5.
2.11) Any of Embodiments 2.0 et seq., wherein Form 2 exhibits an XRPD pattern comprising d-spacing (Å) values of 19.9, 16.2, 14.7, 12.1, 10.4, 9.9, 9.4, 8.6, 8.2, 7.8, 7.2, 7.0, 6.3, 6.0, 5.4, 5.2, 5.0, 4.9, 4.8, 4.6, 4.5, 4.4, 4.3, 4.2, 4.1, 3.9, 3.8, 3.7, and 3.5.
2.12) Any of Embodiments 2.0 et seq., wherein Form 2 exhibits an XRPD pattern comprising at least three, e.g., at least five, e.g., at least ten, e.g., at least fifteen, e.g., at least twenty, e.g., at least twenty-five, e.g., at least twenty-nine, of the following d-spacing (Å) values: 19.9, 16.2, 14.7, 12.6, 12.1, 11.1, 10.4, 9.9, 9.4, 8.6, 8.2, 7.8, 7.6, 7.2, 7.0, 6.8, 6.4, 6.3, 6.0, 5.4, 5.2, 5.0, 4.9, 4.8, 4.6, 4.5, 4.4, 4.3, 4.2, 4.1, 3.9, 3.8, 3.7, 3.6, 3.5, 3.4, and 3.1.
2.13) Any of Embodiments 2.0 et seq., wherein Form 2 exhibits an XRPD pattern comprising d-spacing (Å) values of 19.9, 16.2, 14.7, 12.6, 12.1, 11.1, 10.4, 9.9, 9.4, 8.6, 8.2, 7.8, 7.6, 7.2, 7.0, 6.8, 6.4, 6.3, 6.0, 5.4, 5.2, 5.0, 4.9, 4.8, 4.6, 4.5, 4.4, 4.3, 4.2, 4.1, 3.9, 3.8, 3.7, 3.6, 3.5, 3.4, and 3.1.
2.14) Any of Embodiments 2.0 et seq., wherein Form 2 exhibits an XRPD pattern comprising at least three, e.g., at least five, e.g., at least ten, e.g., at least fifteen, e.g., at least twenty, e.g., at least twenty-five, e.g., at least thirty, of the following d-spacing (Å) values: 19.87, 16.18, 14.68, 12.13, 10.39, 9.92, 9.35, 8.63, 8.24, 7.77, 7.23, 7.03, 6.27, 5.97, 5.36, 5.20, 5.03, 4.90, 4.76, 4.65, 4.61, 4.55, 4.43, 4.33, 4.26, 4.19, 4.06, 3.93, 3.81, 3.66, and 3.53.
2.15) Any of Embodiments 2.0 et seq., wherein Form 2 exhibits an XRPD pattern comprising d-spacing (Å) values of 19.87, 16.18, 14.68, 12.13, 10.39, 9.92, 9.35, 8.63, 8.24, 7.77, 7.23, 7.03, 6.27, 5.97, 5.36, 5.20, 5.03, 4.90, 4.76, 4.65, 4.61, 4.55, 4.43, 4.33, 4.26, 4.19, 4.06, 3.93, 3.81, 3.66, and 3.53.
2.16) Any of Embodiments 2.0 et seq., wherein Form 2 exhibits an XRPD pattern comprising at least three, e.g., at least five, e.g., at least ten, e.g., at least fifteen, e.g., at least twenty, e.g., at least twenty-five, e.g., at least thirty, e.g., at least thirty-one, of the following d-spacing (Å) values: 19.87, 16.18, 14.68, 12.56, 12.13, 11.14, 10.39, 9.92, 9.35, 8.63, 8.24, 7.77, 7.58, 7.23, 7.03, 6.85, 6.45, 6.27, 5.97, 5.36, 5.20, 5.03, 4.90, 4.76, 4.65, 4.61, 4.55, 4.43, 4.33, 4.26, 4.19, 4.06, 3.93, 3.81, 3.66, 3.63, 3.53, 3.49, 3.44, and 3.07.
2.17) Any of Embodiments 2.0 et seq., wherein Form 2 exhibits an XRPD pattern comprising d-spacing (Å) values of 19.87, 16.18, 14.68, 12.56, 12.13, 11.14, 10.39, 9.92, 9.35, 8.63, 8.24, 7.77, 7.58, 7.23, 7.03, 6.85, 6.45, 6.27, 5.97, 5.36, 5.20, 5.03, 4.90, 4.76, 4.65, 4.61, 4.55, 4.43, 4.33, 4.26, 4.19, 4.06, 3.93, 3.81, 3.66, 3.63, 3.53, 3.49, 3.44, and 3.07.
2.18) Any of Embodiments 2.0 et seq., wherein Form 2 exhibits an XRPD pattern comprising at least three, e.g., at least five, e.g., at least ten, e.g., at least fifteen, e.g., at least twenty, e.g., at least twenty-five, e.g., at least thirty, e.g., at least thirty-one, of the d-spacing (Å) values set forth in Table B of Embodiment 2.8.
2.19) Any of Embodiments 2.0 et seq., wherein Form 2 exhibits an XRPD pattern comprising the d-spacing (Å) values set forth in Table B of Embodiment 2.8.
2.20) Any of Embodiments 2.0 et seq., wherein Form 2 exhibits an XRPD pattern comprising at least three, e.g., at least five, e.g., at least ten, e.g., at least fifteen, e.g., at least twenty, e.g., at least twenty-five, e.g., at least thirty, e.g., at least thirty-one, e.g., all of the peaks, of the XRPD shown in FIG. 5, wherein the XRPD is obtained using Cu K radiation.
2.21) Any of Embodiments 2.0 et seq., wherein Form 2 exhibits an XRPD pattern comprising characteristic peaks of the XRPD pattern shown in FIG. 5, wherein the XRPD is obtained using Cu K radiation.
2.22) Any of Embodiments 2.0 et seq., wherein Form 2 exhibits an XRPD pattern comprising representative peaks of the XRPD pattern shown in FIG. 5, wherein the XRPD is obtained using Cu K radiation.
2.23) Any of Embodiments 2.0 et seq., wherein Form 2 exhibits an XRPD pattern corresponding to FIG. 5, wherein the XRPD is obtained using Cu K radiation.
2.24) Any of Embodiments 2.0 et seq., wherein Form 2 exhibits a thermogravimetric analysis (TGA) thermogram comprising weight loss between 20° C. and 300° C., e.g., a weight loss of 18-21 weight %, e.g., a weight loss of 19-20 weight %, e.g., a weight loss of 19.5 weight %. For instance, any of Embodiments 2.0 et seq., wherein Form 2 exhibits a thermogravimetric analysis (TGA) thermogram corresponding to FIG. 6.
2.25) Any of Embodiments 2.0 et seq., wherein Form 2 exhibits a differential analysis thermogram comprising two small endothermic events. For instance, any of Embodiments 2.0 et seq., wherein Form 2 exhibits a differential analysis thermogram corresponding to FIG. 6.
2.26) Any of Embodiments 2.0 et seq., wherein Form 2 exhibits a differential thermal analysis thermogram comprising an endotherm at 71° C., e.g., an endotherm at 71° C. with an onset at 55° C.
2.27) Any of Embodiments 2.0 et seq., wherein Form 2 exhibits a differential thermal analysis thermogram comprising an endotherm at 139° C., e.g., an endotherm at 139° C. with an onset at 126° C.
2.28) Any of Embodiments 2.0 et seq., wherein Form 2 comprises 0.6 equivalents of toluene, e.g., as measured by ¹H NMR.
2.29) Any of Embodiments 2.0 et seq., wherein Form 2 comprises 1.1 equivalents of toluene, e.g., as measured by thermogravimetric analysis (TGA).
2.30) Any of Embodiments 2.0 et seq., wherein preparation of Form 2 comprises mixing, optionally with stirring, shaking, heating, and/or cooling, 3α,7α,11β-trihydroxy-6α-ethyl-5β-cholan-24-oic acid with one or more solvents (e.g., an ICH class 2 or 3 solvent). The one or more solvents may be ones in which 3α,7α,11β-trihydroxy-6α-ethyl-5β-cholan-24-oic acid is soluble (e.g., tert-buty methyl ether and/or methyl ethyl ketone) or less soluble (e.g., poorly soluble) (e.g., water, diisopropyl ether, or a liquid hydrocarbon, e.g., hexane, heptane, and/or toluene).
2.31) Any of Embodiments 2.0 et seq., wherein preparation of Form 2 comprises mixing, optionally with stirring, shaking, heating, and/or cooling, 3α,7α,11β-trihydroxy-6α-ethyl-5β-cholan-24-oic acid with a first solvent in which 3α,7α,11β-trihydroxy-6α-ethyl-5β-cholan-24-oic acid is soluble (e.g., tert-butyl methyl ether and/or ethanol) and a second solvent in which 3α,7α,11β-trihydroxy-6α-ethyl-5β-cholan-24-oic acid is poorly soluble (e.g., diisopropyl ether, or a liquid hydrocarbon, e.g., hexane, heptane, and/or toluene).
2.32) Embodiment 2.31, wherein volume ratio of the first solvent to the second solvent is 1:10 to 1:1, e.g., 1:4 to 1:1, e.g., 1:1.
2.33) Embodiment 2.31 or 2.32, wherein the first solvent is tert-butyl methyl ether or ethanol. For instance, Embodiment 2.31 or 2.32, wherein the first solvent is tert-butyl methyl ether.
2.34) Any of Embodiments 2.31-2.33, wherein the second solvent is hexane, heptane, or toluene. For instance, any of Embodiments 2.31-2.33, wherein the second solvent is heptane. Or, for instance, any of Embodiments 2.31-2.33, wherein the second solvent is toluene.
2.35) Any of Embodiments 2.0-2.30, wherein preparation of Form 2 comprises mixing, optionally with stirring, shaking, heating, and/or cooling, 3α,7α,11β-trihydroxy-6α-ethyl-5β-cholan-24-oic acid with one or more solvents in which 3α,7α,11β-trihydroxy-6α-ethyl-5β-cholan-24-oic acid is poorly soluble (e.g., diisopropyl ether and/or a liquid hydrocarbon, e.g., hexane, heptane, and/or toluene). For instance, any of Embodiments 2.0-2.30, wherein preparation of Form 2 comprises mixing, optionally with stirring, shaking, heating, and/or cooling, 3α,7α,11β-trihydroxy-6α-ethyl-5β-cholan-24-oic acid with two or more solvents in which 3α,7α,11β-trihydroxy-6α-ethyl-5β-cholan-24-oic acid is poorly soluble (e.g., water, diisopropyl ether, and/or a liquid hydrocarbon, e.g., hexane, heptane, and/or toluene). For instance, any of Embodiments 2.0-2.30, wherein preparation of Form 2 comprises mixing, optionally with stirring, shaking, heating, and/or cooling, 3α,7α,11β-trihydroxy-6α-ethyl-5β-cholan-24-oic acid with two solvents in which 3α,7α,11β-trihydroxy-6α-ethyl-5β-cholan-24-oic acid is poorly soluble and the volume ratio of the liquids is 5:1 to 1:1, e.g., 4:1 to 1:1, e.g., 4:1 (e.g., 4:1 diisopropyl ether:hexane or heptane).
2.36) Embodiment 2.35, wherein 3α,7α,11β-trihydroxy-6α-ethyl-5β-cholan-24-oic acid is mixed with diisopropyl ether and hexane or heptane. Or, Embodiment 2.35, wherein 3α,7α,11β-trihydroxy-6α-ethyl-5β-cholan-24-oic acid is mixed with toluene.
2.37) Any of Embodiments 2.0 et seq., wherein preparation of Form 2 comprises mixing, optionally with stirring, shaking, heating, and/or cooling, 3α,7α,11β-trihydroxy-6α-ethyl-5β-cholan-24-oic acid with one or more of tert-butyl methyl ether, ethanol, diisopropyl ether, hexane, heptane, and toluene. For instance, any of Embodiments 2.0 et seq., wherein preparation of Form 2 comprises mixing, optionally with stirring, shaking, heating, and/or cooling, 3α,7α,11β-trihydroxy-6α-ethyl-5β-cholan-24-oic acid with tert-butyl methyl ether, ethanol, or diisopropyl ether and hexane, heptane, or toluene (e.g., diisopropyl ether and heptane or tert-butyl methyl ether and toluene or toluene and ethanol). Or, for instance, any of Embodiments 2.0 et seq., wherein preparation of Form 2 comprises mixing, optionally with stirring, shaking, heating, and/or cooling, 3α,7α,11β-trihydroxy-6α-ethyl-5β-cholan-24-oic acid with tert-butyl methyl ether and toluene.
2.38) Any of Embodiments 2.30-2.37, wherein the mixture of 3α,7α,11β-trihydroxy-6α-ethyl-5β-cholan-24-oic acid and one or more solvents is heated, e.g., above room temperature to 60° C., e.g., between 40° C. to 50° C., e.g., to 40° C. or 50° C. For instance, any of Embodiments 2.30-2.37, wherein the mixture of 3α,7α,11β-trihydroxy-6α-ethyl-5β-cholan-24-oic acid with tert-butyl methyl ether, ethanol, or diisopropyl ether and liquid hydrocarbon (e.g., toluene or heptane) is heated, e.g., to 40° C. or 50° C. Or, for instance, any of Embodiments 2.30-2.37, wherein the mixture of 3α,7α,11β-trihydroxy-6α-ethyl-5β-cholan-24-oic acid and liquid hydrocarbon (e.g., toluene or heptane) is heated, e.g., to 40° C. or 50° C.
2.39) Any of Embodiments 2.30-2.38, wherein the mixture of 3α,7α,11β-trihydroxy-6α-ethyl-5β-cholan-24-oic acid and one or more solvents is thermally cycled, e.g., heated and cooled, e.g., between 50° C. and 0° C., e.g., between 40° C. and 5° C. For instance, any of Embodiments 2.30-2.38, wherein the mixture of 3α,7α,11β-trihydroxy-6α-ethyl-5β-cholan-24-oic acid with tert-butyl methyl ether, ethanol, or diisopropyl ether and liquid hydrocarbon (e.g., toluene or heptane) is thermally cycled, e.g., heated and cooled, e.g., between 40° C. and 5° C.
2.40) Any of Embodiments 2.30-2.39 further comprising allowing the one or more solvents to evaporate, e.g., at ambient conditions or under vacuum.
2.41) Any of Embodiments 2.30-2.40, wherein the mixture is cooled below room temperature, e.g., between −20° C. to 10° C., e.g., between 10° C. and 0° C., e.g., between 5° C. and 0° C.
2.42) Any of Embodiments 2.0 et seq., wherein preparation of Form 2 comprises seeding with Crystalline Form 2 (e.g., any of Embodiments 2.0 et seq.).
2.43) Any of Embodiments 2.30-2.42 further comprising isolating Form 2. 2.44) Embodiment 2.43, wherein Form 2 is isolated by centrifugation and/or filtration.
2.45) Any of Embodiments 2.0 et seq., wherein Form 2 is made by any of Process 2.0 et seq., vide infra.
2.46) Any of Embodiments 2.0 et seq., wherein Form 2 is made as described in any of the examples that produce Form 2.
2.47) Any of Embodiments 2.0 et seq., wherein Form 2 is a toluene solvate.
2.48) Any of Embodiments 2.0 et seq., wherein each of the 2-theta (°) values of the XRPD pattern have an acceptable deviation of ±0.2°.

Another embodiment of the present disclosure provides a Crystalline Form 3 (or Form 3) of Compound 1:

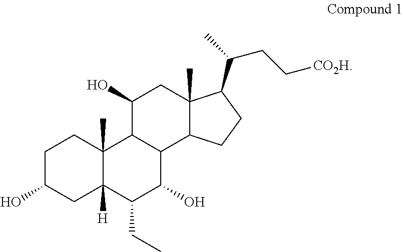 [SMILES: CC[C@H]1[C@@H](O)C2C3CC[C@H]([C@H](C)CCC(=O)O)[C@@]3(C)C[C@H](O)C2[C@@]2(C)CC[C@@H](O)C[C@@H]12]

Further provided are the following embodiments:
3.0) In one of the embodiments, provided is a Crystalline Form 3 (or Form 3) of Compound 1, wherein Form 3 exhibits an XRPD pattern comprising at least three, e.g., at least five, e.g., at least ten, of the following 2-theta (°) values: 5.8, 8.5, 9.1, 9.8, 10.4, 13.0, 15.0, 16.1, 17.5, 18.1, and 22.9, wherein the XRPD is obtained using Cu K radiation
3.1) Embodiment 3.0, wherein Form 3 exhibits an XRPD pattern comprising 2-theta (°) values of 5.8, 8.5, 9.1, 9.8, 10.4, 13.0, 15.0, 16.1, 17.5, 18.1, and 22.9, wherein the XRPD is obtained using Cu K radiation.
3.2) Embodiment 3.0 or 3.1, wherein Form 3 exhibits an XRPD pattern comprising at least three, e.g., at least five, e.g., at least ten, e.g., at least eleven, of the following 2-theta (°) values: 4.1, 5.8, 8.5, 9.1, 9.8, 10.4, 12.1, 13.0, 14.2, 15.0, 16.1, 17.5, 18.1, 19.0, 22.9, 24.9, and 26.2, wherein the XRPD is obtained using Cu K radiation.
3.3) Any of Embodiments 3.0, 3.1, or 3.2, wherein Form 3 exhibits an XRPD pattern comprising 2-theta (°) values of 4.1, 5.8, 8.5, 9.1, 9.8, 10.4, 12.1, 13.0, 14.2, 15.0, 16.1, 17.5, 18.1, 19.0, 22.9, 24.9, and 26.2, wherein the XRPD is obtained using Cu K radiation.
3.4) Any of Embodiments 3.0 et seq., wherein Form 3 exhibits an XRPD pattern comprising at least three, e.g., at least five, e.g., at least ten, of the following 2-theta (°) values: 5.84, 8.51, 9.08, 9.84, 10.37, 13.02, 15.02, 16.09, 17.48, 18.12, and 22.93, wherein the XRPD is obtained using Cu K radiation.
3.5) Any of Embodiments 3.0 et seq., wherein Form 3 exhibits an XRPD pattern comprising 2-theta (°) values of 5.84, 8.51, 9.08, 9.84, 10.37, 13.02, 15.02, 16.09, 17.48, 18.12, and 22.93, wherein the XRPD is obtained using Cu K radiation.
3.6) Any of Embodiments 3.0 et seq., wherein Form 3 exhibits an XRPD pattern comprising at least three, e.g., at least five, e.g., at least ten, e.g., at least eleven, of the following 2-theta (°) values: 4.13, 5.84, 8.51, 9.08, 9.84, 10.37, 12.06, 13.02, 14.25, 15.02, 16.09, 17.48, 18.12, 19.04, 22.93, 24.91, and 26.19, wherein the XRPD is obtained using Cu K radiation.
3.7) Any of Embodiments 3.0 et seq., wherein Form 3 exhibits an XRPD pattern comprising 2-theta (°) values of 4.13, 5.84, 8.51, 9.08, 9.84, 10.37, 12.06, 13.02, 14.25, 15.02, 16.09, 17.48, 18.12, 19.04, 22.93, 24.91, and 26.19, wherein the XRPD is obtained using Cu K radiation.
3.8) Any of Embodiments 3.0 et seq., wherein Form 3 exhibits an XRPD pattern comprising at least three, e.g., at least five, e.g., at least ten, e.g., at least eleven, of the 2-theta (°) values set forth in Table C below:

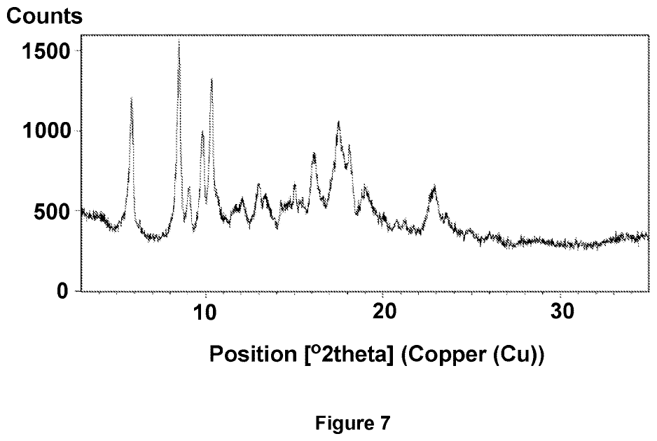

TABLE C

| No. | Pos. [°2θ] | d-spacing [Å] | Rel. Int. [%] |
|---|---|---|---|
| 1 | 4.131 | 21.389 | 10.65 |
| 2 | 5.840 | 15.134 | 71.46 |
| 3 | 8.513 | 10.387 | 100.00 |
| 4 | 9.076 | 9.744 | 24.77 |
| 5 | 9.843 | 8.986 | 53.47 |
| 6 | 10.366 | 8.534 | 80.90 |
| 7 | 12.063 | 7.337 | 17.45 |
| 8 | 13.024 | 6.798 | 25.04 |
| 9 | 14.246 | 6.217 | 14.20 |
| 10 | 15.021 | 5.898 | 23.32 |
| 11 | 16.092 | 5.508 | 38.86 |
| 12 | 17.479 | 5.074 | 52.71 |
| 13 | 18.121 | 4.896 | 41.40 |
| 14 | 19.036 | 4.662 | 19.82 |
| 15 | 22.933 | 3.878 | 23.13 |
| 16 | 24.909 | 3.575 | 4.07 |
| 17 | 26.191 | 3.403 | 3.04, |

wherein the XRPD is obtained using Cu K radiation.
3.9) Any of Embodiments 3.0 et seq., wherein Form 3 exhibits an XRPD pattern comprising the 2-theta (°) values set forth in Table C of Embodiment 3.8, wherein the XRPD is obtained using Cu K radiation.
3.10) Any of Embodiments 3.0 et seq., wherein Form 3 exhibits an XRPD pattern comprising at least three, e.g., at least five, e.g., at least ten, of the following d-spacing (Å) values: 15.1, 10.4, 9.7, 9.0, 8.5, 6.8, 5.9, 5.5, 5.1, 4.9, and 3.9.
3.11) Any of Embodiments 3.0 et seq., wherein Form 3 exhibits an XRPD pattern comprising d-spacing (Å) values of 15.1, 10.4, 9.7, 9.0, 8.5, 6.8, 5.9, 5.5, 5.1, 4.9, and 3.9.
3.12) Any of Embodiments 3.0 et seq., wherein Form 3 exhibits an XRPD pattern comprising at least three, e.g., at least five, e.g., at least ten, e.g., at least eleven, of the following d-spacing (Å) values: 21.4, 15.1, 10.4, 9.7, 9.0, 8.5, 7.3, 6.8, 6.2, 5.9, 5.5, 5.1, 4.9, 4.7, 3.9, 3.6, and 3.4.
3.13) Any of Embodiments 3.0 et seq., wherein Form 3 exhibits an XRPD pattern comprising d-spacing (Å) values of 21.4, 15.1, 10.4, 9.7, 9.0, 8.5, 7.3, 6.8, 6.2, 5.9, 5.5, 5.1, 4.9, 4.7, 3.9, 3.6, and 3.4.
3.14) Any of Embodiments 3.0 et seq., wherein Form 3 exhibits an XRPD pattern comprising at least three, e.g., at least five, e.g., at least ten, e.g., at least eleven, of the following d-spacing (Å) values: 15.13, 10.39, 9.74, 8.99, 8.53, 6.80, 5.90, 5.51, 5.07, 4.90, and 3.88.
3.15) Any of Embodiments 3.0 et seq., wherein Form 3 exhibits an XRPD pattern comprising d-spacing (Å) values of 15.13, 10.39, 9.74, 8.99, 8.53, 6.80, 5.90, 5.51, 5.07, 4.90, and 3.88.
3.16) Any of Embodiments 3.0 et seq., wherein Form 3 exhibits an XRPD pattern comprising at least three, e.g., at least five, e.g., at least ten, e.g., at least eleven, of the following d-spacing (Å) values: 21.39, 15.13, 10.39, 9.74, 8.99, 8.53, 7.34, 6.80, 6.22, 5.90, 5.51, 5.07, 4.90, 4.66, 3.88, 3.58, and 3.40.
3.17) Any of Embodiments 3.0 et seq., wherein Form 3 exhibits an XRPD pattern comprising d-spacing (Å) values of 21.39, 15.13, 10.39, 9.74, 8.99, 8.53, 7.34, 6.80, 6.22, 5.90, 5.51, 5.07, 4.90, 4.66, 3.88, 3.58, and 3.40.
3.18) Any of Embodiments 3.0 et seq., wherein Form 3 exhibits an XRPD pattern comprising at least three, e.g., at least five, e.g., at least ten, e.g., at least eleven, of the d-spacing (Å) values set forth in Table C of Embodiment 3.8.
3.19) Any of Embodiments 3.0 et seq., wherein Form 3 exhibits an XRPD pattern comprising the d-spacing (Å) values set forth in Table C of Embodiment 3.8.
3.20) Any of Embodiments 3.0 et seq., wherein Form 3 comprises 0.3 equivalents of toluene, e.g., as measured by ¹H NMR.
3.21) Any of Embodiments 3.0 et seq., wherein Form 3 exhibits an XRPD pattern comprising at least three, e.g., at least five, e.g., at least ten, e.g., at least eleven, e.g., all of the peaks, of the XRPD shown in FIG. 7, wherein the XRPD is obtained using Cu K radiation.
3.22) Any of Embodiments 3.0 et seq., wherein Form 3 exhibits an XRPD pattern comprising characteristic peaks of the XRPD pattern shown in FIG. 7, wherein the XRPD is obtained using Cu K radiation.
3.23) Any of Embodiments 3.0 et seq., wherein Form 3 exhibits an XRPD pattern comprising representative peaks of the XRPD pattern shown in FIG. 7, wherein the XRPD is obtained using Cu K radiation.
3.24) Any of Embodiments 3.0 et seq., wherein Form 3 exhibits an XRPD pattern corresponding to FIG. 7, wherein the XRPD is obtained using Cu K radiation.
3.25) Any of Embodiments 3.0 et seq., wherein preparation of Form 3 comprises mixing, optionally with stirring, shaking, heating, and/or cooling, 3α,7α,11β-trihydroxy-6α-ethyl-5β-cholan-24-oic acid with one or more solvents (e.g., an ICH class 2 or 3 solvent). The one or more solvents may be ones in which 3α,7α,11β-trihydroxy-6α-ethyl-5β-cholan-24-oic acid is soluble (e.g., tert-buty methyl ether and/or methyl ethyl ketone) or less soluble (e.g., poorly soluble) (e.g., water, diisopropyl ether, or a liquid hydrocarbon, e.g., hexane, heptane, and/or toluene).
3.26) Any of Embodiments 3.0 et seq., wherein preparation of Form 3 comprises mixing, optionally with stirring, shaking, heating, and/or cooling, 3α,7α,11β-trihydroxy-6α-ethyl-5β-cholan-24-oic acid with a solvent in which 3α,7α,11β-trihydroxy-6α-ethyl-5β-cholan-24-oic acid is soluble (e.g., tert-butyl methyl ether) and a second solvent in which 3α,7α,11β-trihydroxy-6α-ethyl-5β-cholan-24-oic acid is poorly soluble (e.g., a liquid hydrocarbon, e.g., toluene).
3.27) Embodiment 3.26, wherein volume ratio of the first solvent to the second solvent is 1:20 to 1:1, e.g., 1:10 to 1:1, e.g., 1:4 to 1:1, e.g., 1:4.
3.28) Embodiment 3.26 or 3.27, wherein the first solvent is tert-butyl methyl ether.
3.29) Any of Embodiments 3.26-3.28, wherein the second solvent is toluene.
3.30) Any of Embodiments 3.0-3.25, wherein preparation of Form 3 comprises mixing, optionally with stirring, shaking, heating, and/or cooling, 3α,7α,11β-trihydroxy-6α-ethyl-5β-cholan-24-oic acid with one or more solvents in which 3α,7α,11β-trihydroxy-6α-ethyl-5β-cholan-24-oic acid is poorly soluble (e.g., diisopropyl ether, and a liquid hydrocarbon, e.g., hexane, heptane, and/or toluene). For instance, any of Embodiments 3.0-3.25, wherein preparation of Form 3 comprises mixing, optionally with stirring, shaking, heating, and/or cooling, 3α,7α,11β-trihydroxy-6α-ethyl-5β-cholan-24-oic acid with two or more solvents in which 3α,7α,11β-trihydroxy-6α-ethyl-5β-cholan-24-oic acid is poorly soluble (e.g., water, diisopropyl ether, and/or a liquid hydrocarbon, e.g., hexane, heptane, and/or toluene). For instance, any of Embodiments 3.0-3.25, wherein preparation of Form 3 comprises mixing, optionally with stirring, shaking, heating, and/or cooling, 3α,7α,11β-trihydroxy-6α-ethyl-5β-cholan-24-oic acid with two solvents in which 3α,7α,11β-trihydroxy-6α-ethyl-5β-cholan-24-oic acid is poorly soluble and the volume ratio of the liquids is 5:1 to 1:1, e.g., 1:1 e.g., 1:1 diisopropyl ether:hexane or heptane).
3.31) Embodiment 3.30, wherein 3α,7α,11β-trihydroxy-6α-ethyl-5β-cholan-24-oic acid is mixed with diisopropyl ether and hexane or heptane.
3.32) Any of Embodiments 3.0 et seq., wherein preparation of Form 3 comprises mixing, optionally with stirring, shaking, heating, and/or cooling, 3α,7α,11β-trihydroxy-6α-ethyl-5β-cholan-24-oic acid with one or more of tert-butyl methyl ether, diisopropyl ether, hexane, heptane, and toluene. For instance, any of Embodiments 3.0 et seq., wherein preparation of Form 3 comprises mixing, optionally with stirring, shaking, heating, and/or cooling, 3α,7α,11β-trihydroxy-6α-ethyl-5β-cholan-24-oic acid with tert-butyl methyl ether and toluene. Or, for instance, any of Embodiments 3.0 et seq., wherein preparation of Form 3 comprises mixing, optionally with stirring, shaking, heating, and/or cooling, 3α,7α,11β-trihydroxy-6α-ethyl-5β-cholan-24-oic acid with diisopropyl ether and hexane or heptane.
3.33) Any of Embodiments 3.25-3.32, wherein the mixture of 3α,7α,11β-trihydroxy-6α-ethyl-5β-cholan-24-oic acid and one or more solvents is heated, e.g., above room temperature to 60° C., e.g., between 40° C. to 50° C., e.g., to 40° C. or 50° C. For instance, any of Embodiments 3.25-3.32, wherein the mixture of 3α,7α,11β-trihydroxy-6α-ethyl-5β-cholan-24-oic acid with tert-butyl methyl ether or diisopropyl and liquid hydrocarbon (e.g., hexane, heptane, or toluene) is heated, e.g., to 40° C. or 50° C.
3.34) Any of Embodiments 3.25-3.33, wherein the mixture of 3α,7α,11β-trihydroxy-6α-ethyl-5β-cholan-24-oic acid and one or more solvents is thermally cycled, e.g., heated and cooled, e.g., between 50° C. and 0° C., e.g., between 40° C. and 5° C. For instance, any of Embodiments 3.25-3.33, wherein the mixture of 3α,7α,11β-trihydroxy-6α-ethyl-5β-cholan-24-oic acid with tert-butyl methyl ether or diisopropyl and liquid hydrocarbon (e.g., hexane, heptane, or toluene) is thermally cycled, e.g., heated and cooled, e.g., between 40° C. and 5° C.
3.35) Any of Embodiments 3.25-3.34 further comprising allowing the one or more solvents to evaporate, e.g., at ambient conditions or under vacuum.
3.36) Any of Embodiments 3.25-3.35, wherein the mixture is cooled below room temperature, e.g., between −20° C. to 10° C., e.g., between 10° C. and 0° C., e.g., between 5° C. and 0° C.
3.37) Any of Embodiments 3.0 et seq., wherein preparation of Form 3 comprises drying Crystalline Form 2.
3.38) Any of Embodiments 3.0 et seq., wherein preparation of Form 3 comprises seeding with Crystalline Form 3 (e.g., any of Embodiments 3.0 et seq.).
3.39) Any of Embodiments 3.25-3.38 further comprising isolating Form 3.
3.40) Embodiment 3.39, wherein Form 3 is isolated by centrifugation and/or filtration.
3.41) Any of Embodiments 3.0 et seq., wherein Form 3 is made by any of Process 3 et seq., vide infra.
3.42) Any of Embodiments 3.0 et seq., wherein Form 3 is made as described in any of the examples that produce Form 3.
3.43) Any of Embodiments 3.0 et seq., wherein Form 3 is a toluene solvate.
3.44) Any of Embodiments 3.0 et seq., wherein each of the 2-theta (°) values of the XRPD pattern have an acceptable deviation of ±0.2°.

Another embodiment of the present disclosure provides a Crystalline Form 4 (or Form 4) of Compound 1:

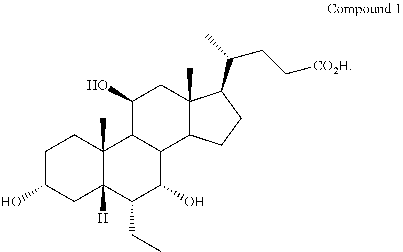 [SMILES: CC[C@H]1[C@@H](O)C2C3CC[C@H]([C@H](C)CCC(=O)O)[C@@]3(C)C[C@H](O)C2[C@@]2(C)CC[C@@H](O)C[C@@H]12]

Further provided are the following embodiments:
4.0) In one of the embodiments, provided is a Crystalline Form 4 (or Form 4) of Compound 1, wherein Form 4 exhibits an XRPD pattern comprising at least three, e.g., at least five, e.g., at least ten, e.g., at least fifteen, of the following 2-theta (°) values: 5.8, 6.2, 7.4, 9.5, 11.7, 12.4, 14.2, 14.5, 15.1, 15.6, 16.0, 17.0, 17.4, 18.2, 19.0, and 20.8, wherein the XRPD is obtained using Cu K radiation.
4.1) Embodiment 4.0, wherein Form 4 exhibits an XRPD pattern comprising 2-theta (°) values of 5.8, 6.2, 7.4, 9.5, 11.7, 12.4, 14.2, 14.5, 15.1, 15.6, 16.0, 17.0, 17.4, 18.2, 19.0, and 20.8, wherein the XRPD is obtained using Cu K radiation.
4.2) Embodiment 4.0 or 4.1, wherein Form 4 exhibits an XRPD pattern comprising at least three, e.g., at least five, e.g., at least ten, e.g., at least fifteen, e.g., at least sixteen, of the following 2-theta (°) values: 5.8, 6.2, 7.4, 9.5, 11.7, 12.4, 14.2, 14.5, 15.1, 15.6, 16.0, 17.0, 17.4, 18.2, 19.0, 20.8, 23.5, 24.3, and 25.5, wherein the XRPD is obtained using Cu K radiation.
4.3) Any of Embodiments 4.0, 4.1, or 4.2, wherein Form 4 exhibits an XRPD pattern comprising 2-theta (°) values of 5.8, 6.2, 7.4, 9.5, 11.7, 12.4, 14.2, 14.5, 15.1, 15.6, 16.0, 17.0, 17.4, 18.2, 19.0, 20.8, 23.5, 24.3, and 25.5, wherein the XRPD is obtained using Cu K radiation.
4.4) Any of Embodiments 4.0 et seq., wherein Form 4 exhibits an XRPD pattern comprising at least three, e.g., at least five, e.g., at least ten, e.g., at least fifteen, of the following 2-theta (°) values: 5.79, 6.23, 7.39, 9.51, 11.68, 12.39, 14.20, 14.49, 15.12, 15.60, 16.04, 16.96, 17.39, 18.16, 18.98, and 20.79, wherein the XRPD is obtained using Cu K radiation.
4.5) Any of Embodiments 4.0 et seq., wherein Form 4 exhibits an XRPD pattern comprising 2-theta (°) values of 5.79, 6.23, 7.39, 9.51, 11.68, 12.39, 14.20, 14.49, 15.12, 15.60, 16.04, 16.96, 17.39, 18.16, 18.98, and 20.79, wherein the XRPD is obtained using Cu K radiation.
4.6) Any of Embodiments 4.0 et seq., wherein Form 4 exhibits XRPD pattern comprising at least three, e.g., at least five, e.g., at least ten, e.g., at least fifteen, e.g., at least sixteen, of the following 2-theta (°) values: 5.79, 6.23, 7.39, 9.51, 11.68, 12.39, 14.20, 14.49, 15.12, 15.60, 16.04, 16.96, 17.39, 18.16, 18.98, 20.79, 23.53, 24.29, and 25.52, wherein the XRPD is obtained using Cu K radiation.
4.7) Any of Embodiments 4.0 et seq., wherein Form 4 exhibits an XRPD pattern comprising 2-theta (°) values of 5.79, 6.23, 7.39, 9.51, 11.68, 12.39, 14.20, 14.49, 15.12, 15.60, 16.04, 16.96, 17.39, 18.16, 18.98, 20.79, 23.53, 24.29, and 25.52, wherein the XRPD is obtained using Cu K radiation.
4.8) Any of Embodiments 4.0 et seq., wherein Form 4 exhibits an XRPD pattern comprising at least three, e.g., at least five, e.g., at least ten, e.g., at least fifteen, e.g., at least sixteen, of the 2-theta (°) values set forth in Table D below:

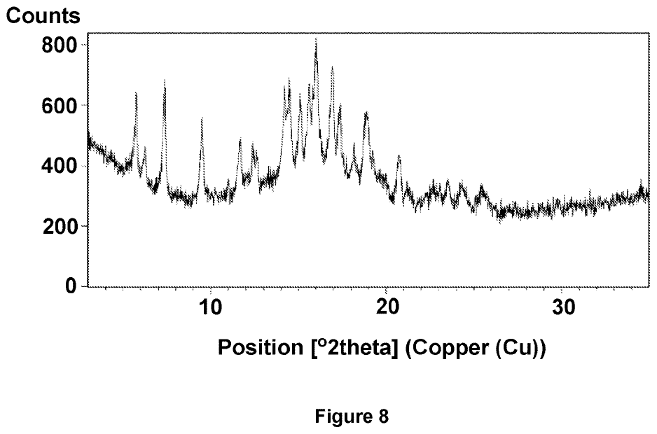

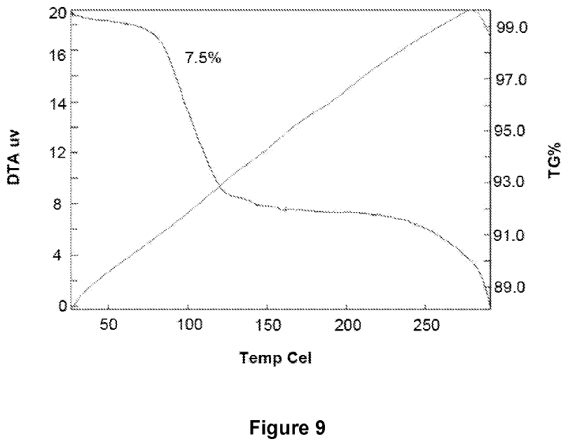

TABLE D

| No. | Pos. [°2θ] | d-spacing [Å] | Rel. Int. [%] |
|---|---|---|---|
| 1 | 5.789 | 15.267 | 54.95 |
| 2 | 6.230 | 14.188 | 23.17 |
| 3 | 7.388 | 11.966 | 81.17 |
| 4 | 9.514 | 9.296 | 55.26 |
| 5 | 11.677 | 7.579 | 40.27 |
| 6 | 12.386 | 7.146 | 30.02 |
| 7 | 14.203 | 6.236 | 72.41 |
| 8 | 14.494 | 6.112 | 73.17 |
| 9 | 15.117 | 5.861 | 59.36 |
| 10 | 15.597 | 5.682 | 73.99 |
| 11 | 16.039 | 5.526 | 100.00 |
| 12 | 16.964 | 5.227 | 88.59 |
| 13 | 17.392 | 5.099 | 58.37 |
| 14 | 18.164 | 4.884 | 28.51 |
| 15 | 18.975 | 4.677 | 56.97 |
| 16 | 20.789 | 4.273 | 29.22 |
| 17 | 23.528 | 3.781 | 18.53 |
| 18 | 24.294 | 3.664 | 16.96 |
| 19 | 25.515 | 3.491 | 17.35, |

wherein the XRPD is obtained using Cu K radiation.
4.9) Any of Embodiments 4.0 et seq., wherein Form 4 exhibits an XRPD pattern comprising the 2-theta (°) values set forth in Table D of Embodiment 4.8, wherein the XRPD is obtained using Cu K radiation.
4.10) Any of Embodiments 4.0 et seq., wherein Form 4 exhibits an XRPD pattern comprising at least three, e.g., at least five, e.g., at least ten, e.g., at least fifteen, of the following d-spacing (Å) values: 15.3, 14.2, 12.0, 9.3, 7.6, 7.1, 6.2, 6.1, 5.9, 5.7, 5.5, 5.2, 5.1, 4.9, 4.7, and 4.3.
4.11) Any of Embodiments 4.0 et seq., wherein Form 4 exhibits an XRPD pattern comprising d-spacing (Å) values of 15.3, 14.2, 12.0, 9.3, 7.6, 7.1, 6.2, 6.1, 5.9, 5.7, 5.5, 5.2, 5.1, 4.9, 4.7, and 4.3.
4.12) Any of Embodiments 4.0 et seq., wherein Form 4 exhibits an XRPD pattern comprising at least three, e.g., at least five, e.g., at least ten, e.g., at least fifteen, e.g., at least sixteen, of the following d-spacing (Å) values: 15.3, 14.2, 12.0, 9.3, 7.6, 7.1, 6.2, 6.1, 5.9, 5.7, 5.5, 5.2, 5.1, 4.9, 4.7, 4.3, 3.8, 3.7, and 3.5.
4.13) Any of Embodiments 4.0 et seq., wherein Form 4 exhibits an XRPD pattern comprising d-spacing (Å) values of 15.3, 14.2, 12.0, 9.3, 7.6, 7.1, 6.2, 6.1, 5.9, 5.7, 5.5, 5.2, 5.1, 4.9, 4.7, 4.3, 3.8, 3.7, and 3.5.
4.14) Any of Embodiments 4.0 et seq., wherein Form 4 exhibits an XRPD pattern comprising at least three, e.g., at least five, e.g., at least ten, e.g., at least fifteen, of the following d-spacing (Å) values: 15.27, 14.19, 11.97, 9.30, 7.58, 7.15, 6.24, 6.11, 5.86, 5.68, 5.53, 5.23, 5.10, 4.88, 4.68, and 4.27.
4.15) Any of Embodiments 4.0 et seq., wherein Form 4 exhibits an XRPD pattern comprising d-spacing (Å) values of 15.27, 14.19, 11.97, 9.30, 7.58, 7.15, 6.24, 6.11, 5.86, 5.68, 5.53, 5.23, 5.10, 4.88, 4.68, and 4.27.
4.16) Any of Embodiments 4.0 et seq., wherein Form 4 exhibits an XRPD pattern comprising at least three, e.g., at least five, e.g., at least ten, e.g., at least fifteen, e.g., at least sixteen, of the following d-spacing (Å) values: 15.27, 14.19, 11.97, 9.30, 7.58, 7.15, 6.24, 6.11, 5.86, 5.68, 5.53, 5.23, 5.10, 4.88, 4.68, 4.27, 3.78, 3.66, and 3.49.
4.17) Any of Embodiments 4.0 et seq., wherein Form 4 exhibits an XRPD pattern comprising d-spacing (Å) values of 15.27, 14.19, 11.97, 9.30, 7.58, 7.15, 6.24, 6.11, 5.86, 5.68, 5.53, 5.23, 5.10, 4.88, 4.68, 4.27, 3.78, 3.66, and 3.49.
4.18) Any of Embodiments 4.0 et seq., wherein Form 4 exhibits XRPD pattern comprising at least three, e.g., at least five, e.g., at least ten, e.g., at least fifteen, e.g., at least sixteen, of the d-spacing (Å) values set forth in Table D of Embodiment 4.8.
4.19) Any of Embodiments 4.0 et seq., wherein Form 4 exhibits an XRPD pattern comprising the d-spacing (Å) values set forth in Table D of Embodiment 4.8.
4.20) Any of Embodiments 4.0 et seq., wherein Form 4 exhibits an XRPD pattern comprising at least three, e.g., at least five, e.g., at least ten, e.g., at least fifteen, e.g., at least sixteen, e.g., all of the peaks, of the XRPD shown in FIG. 8, wherein the XRPD is obtained using Cu K radiation.
4.21) Any of Embodiments 4.0 et seq., wherein Form 4 exhibits an XRPD pattern comprising characteristic peaks of the XRPD pattern shown in FIG. 8, wherein the XRPD is obtained using Cu K radiation.
4.22) Any of Embodiments 4.0 et seq., wherein Form 4 exhibits an XRPD pattern comprising representative peaks of the XRPD pattern shown in FIG. 8, wherein the XRPD is obtained using Cu K radiation.
4.23) Any of Embodiments 4.0 et seq., wherein Form 4 exhibits an XRPD pattern corresponding to FIG. 8, wherein the XRPD is obtained using Cu K radiation.
4.24) Any of Embodiments 4.0 et seq., wherein Form 4 exhibits a thermogravimetric analysis (TGA) thermogram comprising weight loss between 20° C. and 160° C., e.g., a weight loss of 6-9 weight %, e.g., a weight loss of 7-8 weight %, e.g., a weight loss of 7.5 weight %. For instance, any of Embodiments 4.0 et seq., wherein Form 4 exhibits a thermogravimetric analysis (TGA) thermogram corresponding to FIG. 9.
4.25) Any of Embodiments 4.0 et seq., wherein Form 4 exhibits a differential thermal analysis thermogram comprising no endothermic or exothermic events. For instance, any of Embodiments 4.0 et seq., wherein Form 4 exhibits a differential analysis thermogram corresponding to FIG. 9.
4.26) Any of Embodiments 4.0 et seq., wherein Form 4 comprises 0.3 equivalents of diisopropyl ether, e.g., as measured by ¹H NMR.
4.27) Any of Embodiments 4.0 et seq., wherein Form 4 comprises 0.35 equivalents of diisopropyl ether, e.g., as measured by thermogravimetric analysis (TGA).
4.28) Any of Embodiments 4.0 et seq., wherein preparation of Form 4 comprises mixing, optionally with stirring, shaking, heating, and/or cooling, 3α,7α,11β-trihydroxy-6α-ethyl-5β-cholan-24-oic acid with one or more solvents (e.g., an ICH class 2 or 3 solvent). The one or more solvents may be ones in which 3α,7α,11β-trihydroxy-6α-ethyl-5β-cholan-24-oic acid is soluble (e.g., tert-buty methyl ether and/or methyl ethyl ketone) or less soluble (e.g., poorly soluble) (e.g., water, diisopropyl ether, or a liquid hydrocarbon, e.g., hexane, heptane, and/or toluene).
4.29) Any of Embodiments 4.0 et seq., wherein preparation of Form 4 comprises mixing, optionally with stirring, shaking, heating, and/or cooling, 3α,7α,11β-trihydroxy-6α-ethyl-5β-cholan-24-oic acid with one or more solvents in which 3α,7α,11β-trihydroxy-6α-ethyl-5β-cholan-24-oic acid is poorly soluble (e.g., diisopropyl ether, and/or a liquid hydrocarbon, e.g., hexane and/or heptane). For instance, any of Embodiments 4.0 et seq., wherein preparation of Form 4 comprises mixing, optionally with stirring, shaking, heating, and/or cooling, 3α,7α,11β-trihydroxy-6α-ethyl-5β-cholan-24-oic acid with two or more solvents in which 3α,7α,11β-trihydroxy-6α-ethyl-5β-cholan-24-oic acid is poorly soluble (e.g., diisopropyl ether and/or a liquid hydrocarbon, e.g., hexane and/or heptane). For instance, any of Embodiments 4.0 et seq., wherein preparation of Form 4 comprises mixing, optionally with stirring, shaking, heating, and/or cooling, 3α,7α,11β-trihydroxy-6α-ethyl-5β-cholan-24-oic acid with two solvents in which 3α,7α,11β-trihydroxy-6α-ethyl-5β-cholan-24-oic acid is poorly soluble and the volume ratio of the liquids is 5:1 to 1:1, e.g., 4:1 (e.g., 4:1 diisopropyl ether:hexane or heptane).
4.30) Any of Embodiments 4.0 et seq., wherein preparation of Form 4 comprises mixing, optionally with stirring, shaking, heating, and/or cooling, 3α,7α,11β-trihydroxy-6α-ethyl-5β-cholan-24-oic acid with one or more of diisopropyl ether, hexane, and heptane. For instance, any of Embodiments 4.0 et seq., wherein preparation of Form 4 comprises mixing, optionally with stirring, shaking, heating, and/or cooling, 3α,7α,11β-trihydroxy-6α-ethyl-5β-cholan-24-oic acid with diisopropyl ether and heptane.
4.31) Any of Embodiments 4.28-4.30, wherein the mixture of 3α,7α,11β-trihydroxy-6α-ethyl-5β-cholan-24-oic acid and one or more solvents is heated, e.g., above room temperature to 60° C., e.g., between 40° C. to 50° C., e.g., to 40° C. or 50° C. For instance, any of Embodiments 4.28-4.30, wherein the mixture of 3α,7α,11β-trihydroxy-6α-ethyl-5β-cholan-24-oic acid with tert-butyl methyl ether and liquid hydrocarbon (e.g., heptane) is heated, e.g., to 40° C. or 50° C.
4.32) Any of Embodiments 4.28-4.31 further comprising allowing the one or more solvents to evaporate, e.g., at ambient conditions or under vacuum.
4.33) Any of Embodiments 4.28-4.32, wherein the mixture is cooled below room temperature, e.g., between −20° C. to 10° C., e.g., between 10° C. and 0° C., e.g., between 5° C. and 0° C.
4.34) Any of Embodiments 4.0 et seq., wherein preparation of Form 4 comprises seeding with Crystalline Form 4 (e.g., any of Embodiments 4.0 et seq.).
4.35) Any of Embodiments 4.28-4.34 further comprising isolating Form 4.
4.36) Embodiment 4.35, wherein Form 4 is isolated by centrifugation and/or filtration.
4.37) Any of Embodiments 4.0 et seq., wherein Form 4 is made by any of Process 4 et seq., vide infra.
4.38) Any of Embodiments 4.0 et seq., wherein Form 4 is made as described in any of the examples that produce Form 4.
4.39) Any of Embodiments 4.0 et seq., wherein Form 4 is a diisopropyl ether solvate.
4.40) Any of Embodiments 4.0 et seq., wherein each of the 2-theta (°) values of the XRPD pattern have an acceptable deviation of ±0.2°.

Further provided is a process (Process 1) for making 3α,7α,11β-trihydroxy-6α-ethyl-5β-cholan-24-oic acid (Compound 1) in crystalline form, e.g., for making any of Crystalline Form 1 et seq., Crystalline Form 2 et seq., Crystalline Form 3 et seq., and Crystalline Form 4 et seq. Further provided is Process 1 as follows:
1.1) Process 1, wherein the process comprises mixing, optionally with stirring, shaking, heating, and/or cooling, 3α,7α,11β-trihydroxy-6α-ethyl-5β-cholan-24-oic acid with one or more solvents (e.g., an ICH class 2 or 3 solvent). The one or more solvents may be ones in which 3α,7α,11β-trihydroxy-6α-ethyl-5β-cholan-24-oic acid is soluble (e.g., tert-buty methyl ether and/or methyl ethyl ketone) or less soluble (e.g., poorly soluble) (e.g., water, diisopropyl ether, or a liquid hydrocarbon, e.g., hexane, heptane, and/or toluene).
1.2) Process 1 or 1.1, wherein preparation of Form 1 comprises mixing, optionally with stirring, shaking, heating, and/or cooling, 3α,7α,11β-trihydroxy-6α-ethyl-5β-cholan-24-oic acid with a first solvent in which 3α,7α,11β-trihydroxy-6α-ethyl-5β-cholan-24-oic acid is soluble (e.g., tert-butyl methyl ether and/or methyl ethyl ketone) and a second solvent in which 3α,7α,11β-trihydroxy-6α-ethyl-5β-cholan-24-oic acid is poorly soluble (e.g., water, diisopropyl ether, or a liquid hydrocarbon, e.g., hexane, heptane, and/or toluene).
1.3) Process 1.2, wherein volume ratio of the first solvent to the second solvent is 1:1 to 20:1, e.g., 1:1 to 10:1, e.g., 1:1 to 9:1 e.g., 1:1 to 5:1, e.g., 1:1 to 4:1, e.g., 1:1 to 2.3:1.

For instance, Process 1.2, wherein volume ratio of the first solvent to the second solvent is 4:1. Examples of solvents in which 3α,7α,11β-trihydroxy-6α-ethyl-5β-cholan-24-oic acid is soluble are tert-butyl methyl ether and methyl ethyl ketone.

Examples of solvents in which 3α,7α,11β-trihydroxy-6α-ethyl-5β-cholan-24-oic acid are poorly soluble are hexane, heptane, toluene, and water. Or, Process 1.2, wherein volume ratio of the first solvent to the second solvent is 1:20 to 1:1, e.g., 1:10 to 1:1, e.g., 1:4 to 1:1, e.g., 1:1.
1.4) Process 1.2 or 1.3, wherein the first solvent is tert-butyl methyl ether or methyl ethyl ketone. For instance, Process 1.2 or 1.3, wherein the first solvent is tert-butyl methyl ether.
1.5) Any of Process 1.2-1.4, wherein the second solvent is hexane, heptane, toluene, or water. For instance, any of Process 1.2-1.4, wherein the second solvent is heptane. Or, for instance, any of Process 1.2-1.4, wherein the second solvent is water. Or, for instance, any of Process 1.2-1.4, wherein the second solvent is toluene.
1.6) Any of Process 1 et seq. wherein preparation of Form 1 comprises mixing, optionally with stirring, shaking, heating, and/or cooling, 3α,7α,11β-trihydroxy-6α-ethyl-5β-cholan-24-oic acid with one or more solvents in which 3α,7α,11β-trihydroxy-6α-ethyl-5β-cholan-24-oic acid is poorly soluble (e.g., water, diisopropyl ether, and/or a liquid hydrocarbon, e.g., hexane, heptane, and/or toluene). For instance, any of Process 1 et seq., wherein preparation of Form 1 comprises mixing, optionally with stirring, shaking, heating, and/or cooling, 3α,7α,11β-trihydroxy-6α-ethyl-5β-cholan-24-oic acid with two or more solvents in which 3α,7α,11β-trihydroxy-6α-ethyl-5β-cholan-24-oic acid is poorly soluble (e.g., water, diisopropyl ether, and/or a liquid hydrocarbon, e.g., hexane, heptane, and/or toluene). For instance, any of Process 1 et seq., wherein preparation of Form 1 comprises mixing, optionally with stirring, shaking, heating, and/or cooling, 3α,7α,11β-trihydroxy-6α-ethyl-5β-cholan-24-oic acid with two solvents in which 3α,7α,11β-trihydroxy-6α-ethyl-5β-cholan-24-oic acid is poorly soluble and the volume ratio of the liquids is 5:1 to 1:1, e.g., 1:1 (e.g., 1:1 diisopropyl ether:hexane or heptane).
1.7) Process 1.6, wherein 3α,7α,11β-trihydroxy-6α-ethyl-5β-cholan-24-oic acid is mixed with diisopropyl ether and hexane or heptane.
1.8) Any of Process 1 et seq., wherein preparation of Form 1 comprises mixing, optionally with stirring, shaking, heating, and/or cooling, 3α,7α,11β-trihydroxy-6α-ethyl-5β-cholan-24-oic acid with one or more of tert-butyl methyl ether, methyl ethyl ketone, diisopropyl ether, hexane, heptane, toluene, and water. For instance, any of Process 1 et seq., wherein preparation of Form 1 comprises mixing, optionally with stirring, shaking, heating, and/or cooling, 3α,7α,11β-trihydroxy-6α-ethyl-5β-cholan-24-oic acid with tert-butyl methyl ether, diisopropyl ether, or methyl ethyl ketone and hexane or heptane. Or, for instance, any of Process 1 et seq., wherein preparation of Form 1 comprises mixing, optionally with stirring, shaking, heating, and/or cooling, 3α,7α,11β-trihydroxy-6α-ethyl-5β-cholan-24-oic acid with tert-butyl methyl ether and water (e.g., tert-butyl methyl ether saturated water). Or, for instance, any of Process 1 et seq., wherein preparation of Form 1 comprises mixing, optionally with stirring, shaking, heating, and/or cooling, 3α,7α,11β-trihydroxy-6α-ethyl-5β-cholan-24-oic acid with tert-butyl methyl ether and toluene.
1.9) Any of Process 1.1-1.8, wherein the mixture of 3α,7α,11β-trihydroxy-6α-ethyl-5β-cholan-24-oic acid and one or more solvents is heated, e.g., above room temperature to 60° C., e.g., between 40° C. to 50° C., e.g., to 40° C. or 50° C. For instance, any of Process 1.1-1.8, wherein the mixture of 3α,7α,11β-trihydroxy-6α-ethyl-5β-cholan-24-oic acid with tert-butyl methyl ether and liquid hydrocarbon (e.g., heptane) is heated, e.g., to 40° C. or 50° C.
1.10) Any of Process 1.1-1.9, wherein the mixture of 3α,7α,11β-trihydroxy-6α-ethyl-5β-cholan-24-oic acid and one or more solvents is thermally cycled, e.g., heated and cooled, e.g., between 50° C. and 0° C., e.g., between 40° C. and 5° C. For instance, any of Process 1.1-1.9, wherein the mixture of 3α,7α,11β-trihydroxy-6α-ethyl-5β-cholan-24-oic acid with tert-butyl methyl ether and liquid hydrocarbon (e.g., heptane) is thermally cycled, e.g., heated and cooled, e.g., between 40° C. and 5° C.
1.11) Any of Process 1.1-1.10 further comprising allowing the one or more solvents to evaporate, e.g., at ambient conditions or under vacuum.
1.12) Any of Process 1.1-1.11, wherein the mixture is cooled below room temperature, e.g., between −20° C. to 10° C., e.g., between 10° C. and 0° C., e.g., between 5° C. and 0° C.
1.13) Any of Process 1 et seq., wherein preparation of Form 1 comprises seeding with Crystalline Form 1 (e.g., any of Embodiments 1.0 et seq.).
1.14) Any of Process 1 et seq. further comprising isolating Form 1.
1.15) Any of Process 1 et seq., wherein Form 1 is isolated by centrifugation and/or filtration.
1.16) Any of Process 1 et seq., wherein the process is as described in any of the examples that produce Form 1.
1.17) A crystalline form of 3α,7α,11β-trihydroxy-6α-ethyl-5β-cholan-24-oic acid made by any of Process 1 et seq.
1.18) Any of Crystalline Form 1 et seq., Crystalline Form 2 et seq., Crystalline Form 3 et seq., and Crystalline Form 4 et seq., wherein the crystal is made by any of Process 1 et seq.

Further provided is a crystal made by any of Process 1 et seq.

Further provided is a process (Process 2) for making 3α,7α,11β-trihydroxy-6α-ethyl-5β-cholan-24-oic acid (Compound 1) in crystalline form, e.g., for making any of Crystalline Form 1 et seq., Crystalline Form 2 et seq., Crystalline Form 3 et seq., and Crystalline Form 4 et seq. Further provided is Process 2 as follows:
2.1) Process 2, wherein preparation of Form 2 comprises mixing, optionally with stirring, shaking, heating, and/or cooling, 3α,7α,11β-trihydroxy-6α-ethyl-5β-cholan-24-oic acid with one or more solvents (e.g., an ICH class 2 or 3 solvent). The one or more solvents may be ones in which 3α,7α,11β-trihydroxy-6α-ethyl-5β-cholan-24-oic acid is soluble (e.g., tert-buty methyl ether and/or methyl ethyl ketone) or less soluble (e.g., poorly soluble) (e.g., water, diisopropyl ether, or a liquid hydrocarbon, e.g., hexane, heptane, and/or toluene).
2.2) Process 2 or 2.1, wherein preparation of Form 2 comprises mixing, optionally with stirring, shaking, heating, and/or cooling, 3α,7α,11β-trihydroxy-6α-ethyl-5β-cholan-24-oic acid with a first solvent in which 3α,7α,11β-trihydroxy-6α-ethyl-5β-cholan-24-oic acid is soluble (e.g., tert-butyl methyl ether and/or ethanol) and a second solvent in which 3α,7α,11β-trihydroxy-6α-ethyl-5β-cholan-24-oic acid is poorly soluble (e.g., diisopropyl ether, or a liquid hydrocarbon, e.g., hexane, heptane, and/or toluene).
2.3) Process 2.1, wherein volume ratio of the first solvent to the second solvent is 1:10 to 1:1, e.g., 1:4 to 1:1, e.g., 1:1.
2.4) Process 2.2 or 2.3, wherein the first solvent is tert-butyl methyl ether or ethanol. For instance, Process 2.2 or 2.3, wherein the first solvent is tert-butyl methyl ether.
2.5) Any of Process 2.2-2.4, wherein the second solvent is hexane, heptane, or toluene. For instance, any of Process 2.2-2.4, wherein the second solvent is heptane. Or, for instance, any of Process 2.2-2.4, wherein the second solvent is toluene.
2.6) Any of Process 2 et seq., wherein preparation of Form 2 comprises mixing, optionally with stirring, shaking, heating, and/or cooling, 3α,7α,11β-trihydroxy-6α-ethyl-5β-cholan-24-oic acid with one or more solvents in which 3α,7α,11β-trihydroxy-6α-ethyl-5β-cholan-24-oic acid is poorly soluble (e.g., diisopropyl ether and/or a liquid hydrocarbon, e.g., hexane, heptane, and/or toluene). For instance, any of Process 2 et seq., wherein preparation of Form 2 comprises mixing, optionally with stirring, shaking, heating, and/or cooling, 3α,7α,11β-trihydroxy-6α-ethyl-5β-cholan-24-oic acid with two or more solvents in which 3α,7α,11β-trihydroxy-6α-ethyl-5β-cholan-24-oic acid is poorly soluble (e.g., water, diisopropyl ether, and/or a liquid hydrocarbon, e.g., hexane, heptane, and/or toluene). For instance, any of Process 2 et seq., wherein preparation of Form 2 comprises mixing, optionally with stirring, shaking, heating, and/or cooling, 3α,7α,11β-trihydroxy-6α-ethyl-5β-cholan-24-oic acid with two solvents in which 3α,7α,11β-trihydroxy-6α-ethyl-5β-cholan-24-oic acid is poorly soluble and the volume ratio of the liquids is 5:1 to 1:1, e.g., 4:1 to 1:1, e.g., 4:1 (e.g., 4:1 diisopropyl ether:hexane or heptane).
2.7) Process 2.6, wherein 3α,7α,11β-trihydroxy-6α-ethyl-5β-cholan-24-oic acid is mixed with diisopropyl ether and hexane or heptane. Or, Process 2.6, wherein 3α,7α,11β-trihydroxy-6α-ethyl-5β-cholan-24-oic acid is mixed with toluene.
2.8) Any of Process 2 et seq., wherein preparation of Form 2 comprises mixing, optionally with stirring, shaking, heating, and/or cooling, 3α,7α,11β-trihydroxy-6α-ethyl-5β-cholan-24-oic acid with one or more of tert-butyl methyl ether, ethanol, diisopropyl ether, hexane, heptane, and toluene. For instance, any of Process 2 et seq., wherein preparation of Form 2 comprises mixing, optionally with stirring, shaking, heating, and/or cooling, 3α,7α,11β-trihydroxy-6α-ethyl-5β-cholan-24-oic acid with tert-butyl methyl ether, ethanol, or diisopropyl ether and hexane, heptane, or toluene (e.g., diisopropyl ether and heptane or tert-butyl methyl ether and toluene or toluene and ethanol). Or, for instance, any of Process 2 et seq., wherein preparation of Form 2 comprises mixing, optionally with stirring, shaking, heating, and/or cooling, 3α,7α,11β-trihydroxy-6α-ethyl-5β-cholan-24-oic acid with tert-butyl methyl ether and toluene.
2.9) Any of Process 2.1-2.8, wherein the mixture of 3α,7α,11β-trihydroxy-6α-ethyl-5β-cholan-24-oic acid and one or more solvents is heated, e.g., above room temperature to 60° C., e.g., between 40° C. to 50° C., e.g., to 40° C. or 50° C. For instance, any of Process 2.1-2.8, wherein the mixture of 3α,7α,11β-trihydroxy-6α-ethyl-5β-cholan-24-oic acid with tert-butyl methyl ether, ethanol, or diisopropyl ether and liquid hydrocarbon (e.g., toluene or heptane) is heated, e.g., to 40° C. or 50° C. Or, for instance, any of Process 2.1-2.8, wherein the mixture of 3α,7α,11β-trihydroxy-6α-ethyl-5β-cholan-24-oic acid and liquid hydrocarbon (e.g., toluene or heptane) is heated, e.g., to 40° C. or 50° C.
2.10) Any of Process 2.1-2.9, wherein the mixture of 3α,7α,11β-trihydroxy-6α-ethyl-5β-cholan-24-oic acid and one or more solvents is thermally cycled, e.g., heated and cooled, e.g., between 50° C. and 0° C., e.g., between 40° C. and 5° C. For instance, any of Process 2.1-2.9, wherein the mixture of 3α,7α,11β-trihydroxy-6α-ethyl-5 β-cholan-24-oic acid with tert-butyl methyl ether, ethanol, or diisopropyl ether and liquid hydrocarbon (e.g., toluene or heptane) is thermally cycled, e.g., heated and cooled, e.g., between 40° C. and 5° C.
2.11) Any of Process 2.1-2.10 further comprising allowing the one or more solvents to evaporate, e.g., at ambient conditions or under vacuum.
2.12) Any of Process 2.1-2.11, wherein the mixture is cooled below room temperature, e.g., between −20° C. to 10° C., e.g., between 10° C. and 0° C., e.g., between 5° C. and 0° C.
2.13) Any of Process 2 et seq., wherein preparation of Form 2 comprises seeding with Crystalline Form 2 (e.g., any of Embodiments 2.0 et seq.).
2.14) Any of Process 2 et seq. comprising isolating Form 2.
2.15) Any of Process 2 et seq., wherein Form 2 is isolated by centrifugation and/or filtration.
2.16) Any of Process 2 et seq., wherein the process is as described in any of the examples that produce Form 2.
2.17) A crystalline form of 3α,7α,11β-trihydroxy-6α-ethyl-5β-cholan-24-oic acid made by any of Process 2 et seq.
2.18) Any of Crystalline Form 1 et seq., Crystalline Form 2 et seq., Crystalline Form 3 et seq., and Crystalline Form 4 et seq., wherein the crystal is made by any of Process 2 et seq.

Further provided is a crystal made by any of Process 2 et seq.

Further provided is a process (Process 3) for making 3α,7α,11β-trihydroxy-6α-ethyl-5β-cholan-24-oic acid (Compound 1) in crystalline form, e.g., for making any of Crystalline Form 1 et seq., Crystalline Form 2 et seq., Crystalline Form 3 et seq., and Crystalline Form 4 et seq. Further provided is Process 3 as follows:
3.1) Process 3, wherein preparation of Form 3 comprises mixing, optionally with stirring, shaking, heating, and/or cooling, 3α,7α,11β-trihydroxy-6α-ethyl-5β-cholan-24-oic acid with one or more solvents (e.g., an ICH class 2 or 3 solvent). The one or more solvents may be ones in which 3α,7α,11β-trihydroxy-6α-ethyl-5β-cholan-24-oic acid is soluble (e.g., tert-buty methyl ether and/or methyl ethyl ketone) or less soluble (e.g., poorly soluble) (e.g., water, diisopropyl ether, or a liquid hydrocarbon, e.g., hexane, heptane, and/or toluene).
3.2) Process 3 or 3.1, wherein preparation of Form 3 comprises mixing, optionally with stirring, shaking, heating, and/or cooling, 3α,7α,11β-trihydroxy-6α-ethyl-5β-cholan-24-oic acid with a first solvent in which 3α,7α,11β-trihydroxy-6α-ethyl-5β-cholan-24-oic acid is soluble (e.g., tert-butyl methyl ether) and a second solvent in which 3α,7α,11β-trihydroxy-6α-ethyl-5β-cholan-24-oic acid is poorly soluble (e.g., a liquid hydrocarbon, e.g., toluene).
3.3) Process 3.2, wherein volume ratio of the first solvent to the second solvent is 1:20 to 1:1, e.g., 1:10 to 1:1, e.g., 1:4 to 1:1, e.g., 1:4.
3.4) Process 3.2 or 3.3, wherein the first solvent is tert-butyl methyl ether.
3.5) Any of Process 3.2-3.4, wherein the second solvent is toluene.
3.6) Any of Process 3 et seq., wherein preparation of Form 3 comprises mixing, optionally with stirring, shaking, heating, and/or cooling, 3α,7α,11β-trihydroxy-6α-ethyl-5β-cholan-24-oic acid with one or more solvents in which 3α,7α,11β-trihydroxy-6α-ethyl-5β-cholan-24-oic acid is poorly soluble (e.g., diisopropyl ether, and a liquid hydrocarbon, e.g., hexane, heptane, and/or toluene). For instance, any of Process 3 et seq., wherein preparation of Form 3 comprises mixing, optionally with stirring, shaking, heating, and/or cooling, 3α,7α,11β-trihydroxy-6α-ethyl-5β-cholan-24-oic acid with two or more solvents in which 3α,7α,11β-trihydroxy-6α-ethyl-5β-cholan-24-oic acid is poorly soluble (e.g., water, diisopropyl ether, and/or a liquid hydrocarbon, e.g., hexane, heptane, and/or toluene). For instance, any of Process 3 et seq., wherein preparation of Form 3 comprises mixing, optionally with stirring, shaking, heating, and/or cooling, 3α,7α,11β-trihydroxy-6α-ethyl-5β-cholan-24-oic acid with two solvents in which 3α,7α,11β-trihydroxy-6α-ethyl-5β-cholan-24-oic acid is poorly soluble and the volume ratio of the liquids is 5:1 to 1:1, e.g., 1:1 e.g., 1:1 diisopropyl ether:hexane or heptane).
3.7) Process 3.6, wherein 3α,7α,11β-trihydroxy-6α-ethyl-5β-cholan-24-oic acid is mixed with diisopropyl ether and hexane or heptane.
3.8) Any of Process 3 et seq., wherein preparation of Form 3 comprises mixing, optionally with stirring, shaking, heating, and/or cooling, 3α,7α,11β-trihydroxy-6α-ethyl-5β-cholan-24-oic acid with one or more of tert-butyl methyl ether, diisopropyl ether, hexane, heptane, and toluene. For instance, any of Process 3 et seq., wherein preparation of Form 3 comprises mixing, optionally with stirring, shaking, heating, and/or cooling, 3α,7α,11β-trihydroxy-6α-ethyl-5β-cholan-24-oic acid with tert-butyl methyl ether and toluene. Or, for instance, any of Process 3 et seq., wherein preparation of Form 3 comprises mixing, optionally with stirring, shaking, heating, and/or cooling, 3α,7α,11β-trihydroxy-6α-ethyl-5β-cholan-24-oic acid with diisopropyl ether and hexane or heptane.
3.9) Any of Process 3.1-3.8, wherein the mixture of 3α,7α,11β-trihydroxy-6α-ethyl-5β-cholan-24-oic acid and one or more solvents is heated, e.g., above room temperature to 60° C., e.g., between 40° C. to 50° C., e.g., to 40° C. or 50° C. For instance, any of Process 3.1-3.8, wherein the mixture of 3α,7α,11β-trihydroxy-6α-ethyl-5β-cholan-24-oic acid with tert-butyl methyl ether or diisopropyl and liquid hydrocarbon (e.g., hexane, heptane, or toluene) is heated, e.g., to 40° C. or 50° C.
3.10) Any of Process 3.1-3.9, wherein the mixture of 3α,7α,11β-trihydroxy-6α-ethyl-5β-cholan-24-oic acid and one or more solvents is thermally cycled, e.g., heated and cooled, e.g., between 50° C. and 0° C., e.g., between 40° C. and 5° C. For instance, any of Process 3.1-3.9, wherein the mixture of 3α,7α,11β-trihydroxy-6α-ethyl-5β-cholan-24-oic acid with tert-butyl methyl ether or diisopropyl and liquid hydrocarbon (e.g., hexane, heptane, or toluene) is thermally cycled, e.g., heated and cooled, e.g., between 40° C. and 5° C.
3.11) Any of Process 3.1-3.10 further comprising allowing the one or more solvents to evaporate, e.g., at ambient conditions or under vacuum.
3.12) Any of Process 3.1-3.11, wherein the mixture is cooled below room temperature, e.g., between −20° C. to 10° C., e.g., between 10° C. and 0° C., e.g., between 5° C. and 0° C.
3.13) Any of Process 3 et seq., wherein preparation of Form 3 comprises drying Crystalline Form 2.
3.14) Any of Process 3 et seq., wherein preparation of Form 3 comprises seeding with Crystalline Form 3 (e.g., any of Embodiments 3.0 et seq.).
3.15) Any of Process 3 et seq. comprising isolating Form 3.
3.16) Any of Process 3 et seq., wherein Form 3 is isolated by centrifugation and/or filtration.
3.17) Any of Process 3 et seq., wherein the process is as described in any of the examples that produce Form 3.
3.18) A crystalline form of 3α,7α,11β-trihydroxy-6α-ethyl-5β-cholan-24-oic acid made by any of Process 3 et seq.
3.19) Any of Crystalline Form 1 et seq., Crystalline Form 2 et seq., Crystalline Form 3 et seq., and Crystalline Form 4 et seq., wherein the crystal is made by any of Process 3 et seq.

Further provided is a crystal made by any of Process 3 et seq.

Further provided is a process (Process 4) for making 3α,7α,11β-trihydroxy-6α-ethyl-5β-cholan-24-oic acid (Compound 1) in crystalline form, e.g., for making any of Crystalline Form 1 et seq., Crystalline Form 2 et seq., Crystalline Form 3 et seq., and Crystalline Form 4 et seq. Further provided is Process 4 as follows:
4.1) Process 4, wherein preparation of Form 4 comprises mixing, optionally with stirring, shaking, heating, and/or cooling, 3α,7α,11β-trihydroxy-6α-ethyl-5β-cholan-24-oic acid with one or more solvents (e.g., an ICH class 2 or 3 solvent). The one or more solvents may be ones in which 3α,7α,11β-trihydroxy-6α-ethyl-5β-cholan-24-oic acid is soluble (e.g., tert-buty methyl ether and/or methyl ethyl ketone) or less soluble (e.g., poorly soluble) (e.g., water, diisopropyl ether, or a liquid hydrocarbon, e.g., hexane, heptane, and/or toluene).
4.2) Process 4 or 4.1, wherein preparation of Form 4 comprises mixing, optionally with stirring, shaking, heating, and/or cooling, 3α,7α,11β-trihydroxy-6α-ethyl-5β-cholan-24-oic acid with one or more solvents in which 3α,7α,11β-trihydroxy-6α-ethyl-5β-cholan-24-oic acid is poorly soluble (e.g., diisopropyl ether, and/or a liquid hydrocarbon, e.g., hexane and/or heptane). For instance, Process 4 or 4.1, wherein preparation of Form 4 comprises mixing, optionally with stirring, shaking, heating, and/or cooling, 3α,7α,11β-trihydroxy-6α-ethyl-5β-cholan-24-oic acid with two or more solvents in which 3α,7α,11β-trihydroxy-6α-ethyl-5β-cholan-24-oic acid is poorly soluble (e.g., diisopropyl ether and/or a liquid hydrocarbon, e.g., hexane and/or heptane). For instance, Process 4 or 4.1, wherein preparation of Form 4 comprises mixing, optionally with stirring, shaking, heating, and/or cooling, 3α,7α,11 β-trihydroxy-6α-ethyl-5β-cholan-24-oic acid with two solvents in which 3α,7α,11β-trihydroxy-6α-ethyl-5β-cholan-24-oic acid is poorly soluble and the volume ratio of the liquids is 5:1 to 1:1, e.g., 4:1 (e.g., 4:1 diisopropyl ether:hexane or heptane).
4.3) Any of Process 4 et seq., wherein preparation of Form 4 comprises mixing, optionally with stirring, shaking, heating, and/or cooling, 3α,7α,11β-trihydroxy-6α-ethyl-5β-cholan-24-oic acid with one or more of diisopropyl ether, hexane, and heptane. For instance, any of Process 4 et seq., wherein preparation of Form 4 comprises mixing, optionally with stirring, shaking, heating, and/or cooling, 3α,7α,11β-trihydroxy-6α-ethyl-5β-cholan-24-oic acid with diisopropyl ether and heptane.
4.4) Any of Process 4.1-4.3, wherein the mixture of 3α,7α,11β-trihydroxy-6α-ethyl-5β-cholan-24-oic acid and one or more solvents is heated, e.g., above room temperature to 60° C., e.g., between 40° C. to 50° C., e.g., to 40° C. or 50° C. For instance, any of Process 4.1-4.3, wherein the mixture of 3α,7α,11β-trihydroxy-6α-ethyl-5β-cholan-24-oic acid with tert-butyl methyl ether and liquid hydrocarbon (e.g., heptane) is heated, e.g., to 40° C. or 50° C.
4.5) Any of Process 4.1-4.4 further comprising allowing the one or more solvents to evaporate, e.g., at ambient conditions or under vacuum.
4.6) Any of Process 4.1-4.5, wherein the mixture is cooled below room temperature, e.g., between −20° C. to 10° C., e.g., between 10° C. and 0° C., e.g., between 5° C. and 0° C.
4.7) Any of Process 4 et seq., wherein preparation of Form 4 comprises seeding with Crystalline Form 4 (e.g., any of Embodiments 4.0 et seq.).
4.8) Any of Process 4 et seq. comprising isolating Form 4.
4.9) Any of Process 4 et seq., wherein the crystal is isolated by centrifugation and/or filtration.
4.10) Any of Process 4 et seq., wherein the process is as described in any of the examples that produce Form 4.
4.11) A crystalline form of 3α,7α,11β-trihydroxy-6α-ethyl-5β-cholan-24-oic acid made by any of Process 4 et seq.
4.12) Any of Crystalline Form 1 et seq., Crystalline Form 2 et seq., Crystalline Form 3 et seq., and Crystalline Form 4 et seq., wherein the crystal is made by any of Process 4 et seq.

Further provided is a crystal made by any of Process 4 et seq.

Further provided is a pharmaceutical composition (Composition 1), e.g., a pharmaceutical composition as described in any of U.S. Pat. Nos. 9,611,289 and 11,066,437, U.S. Publications Nos. 2018/0256600 and 2020/0164005, and International Publication No. WO 2018/226724, comprising 3α,7α,11β-trihydroxy-6α-ethyl-5β-cholan-24-oic acid (Compound 1) in crystalline form, e.g., any of Crystalline Form 1 et seq., Crystalline Form 2 et seq., Crystalline Form 3 et seq., and Crystalline Form 4 et seq., and optionally a pharmaceutically acceptable excipient.

Further provided is a process (Process 5) for preparing a pharmaceutical composition comprising 3α,7α,11β-trihydroxy-6α-ethyl-5β-cholan-24-oic acid (Compound 1), wherein the process comprises mixing a crystalline form of 3α,7α,11β-trihydroxy-6α-ethyl-5β-cholan-24-oic acid as described herein, e.g., any of Crystalline Form 1 et seq., 2 et seq., 3 et seq., or 4 et seq., and a pharmaceutically acceptable excipient.

Further provided is a process (Process 6) for purifying 3α,7α,11β-trihydroxy-6α-ethyl-5β-cholan-24-oic acid (Compound 1). Further provided is Process 6 as follows:
6.1) Process 6, wherein the process comprises mixing a crystalline form of 3α,7α,11β-trihydroxy-6α-ethyl-5β-cholan-24-oic acid as described herein, e.g., any of Crystalline Form 1 et seq., 2 et seq., 3 et seq., or 4 et seq., with a solvent (e.g., water).
6.2) Process 6 or 6.1, wherein the process comprises mixing a crystalline form of 3α,7α,11β-trihydroxy-6α-ethyl-5β-cholan-24-oic acid as described herein, e.g., any of Crystalline Form 1 et seq., 2 et seq., 3 et seq., or 4 et seq., with a base (e.g., with a base to pH 9). For instance, Process 6 or 6.1, wherein the process comprises mixing a crystalline form of 3α,7α,11β-trihydroxy-6α-ethyl-5β-cholan-24-oic acid as described herein, e.g., any of Crystalline Form 1 et seq., 2 et seq., 3 et seq., or 4 et seq., with an aqueous solution comprising a base (e.g., wherein the aqueous solution comprising a base is added to pH 9). Or, for instance, Process 6 or 6.1, wherein the process comprises mixing a crystalline form of 3α,7α,11β-trihydroxy-6α-ethyl-5β-cholan-24-oic acid as described herein, e.g., any of Crystalline Form 1 et seq., 2 et seq., 3 et seq., or 4 et seq., with a solvent, wherein the solvent is a base.
6.3) Process 6.2, wherein the base has a pKb<1.
6.4) Process 6.2 or 6.3, wherein the base is an alkali hydroxide base. For instance, Process 6.2 or 6.3, wherein the base is one or more of lithium hydroxide, potassium hydroxide, and sodium hydroxide.
6.5) Any of Process 6.2-6.4 wherein the process comprises mixing the crystalline form with an aqueous solution of the base (e.g., a dilute aqueous solution). For instance, any of Process 6.2-6.4, wherein the process comprises mixing the crystalline form with a<1 M aqueous solution of the base, e.g., a<0.5 M aqueous solution of the base. For instance, any of Process 6.2-6.4, wherein the process comprises mixing the crystalline form with an aqueous solution of 0.3 M NaOH.
6.6) Process 6.2-6.5, wherein the process comprises mixing the crystalline form with 1-1.5 equivalents of the base (e.g., with 1-1.2 equivalents of the base, e.g., with 1 equivalent of the base).
6.7) Any of Process 6.1-6.6, wherein the process comprises concentration of the solvent (e.g., the aqueous solution), e.g., concentration of the solvent under vacuum and/or with heating.
6.8) Any of Process 6 et seq., wherein the process comprises mixing 3α,7α,11β-trihydroxy-6α-ethyl-5β-cholan-24-oic acid (e.g., a crystalline form of 3α,7α,11β-trihydroxy-6α-ethyl-5β-cholan-24-oic acid as described herein, e.g., any of Crystalline Form 1 et seq., 2 et seq., 3 et seq., or 4 et seq) with an acid (e.g., with an acid to pH<4, e.g., pH 3.5).
6.9) Process 6.8, wherein the process comprises mixing with 1-1.5 equivalents of the acid (e.g., with 1-1.2 equivalents, e.g., with 1.1 equivalent).
6.10) Process 6.8 or 6.9, wherein the acid has a pKa<0. For instance, Process 6.8 or 6.9, wherein the acid is hydrochloric acid.
6.11) Process 6.8 or 6.9, wherein the mixture is heated (e.g., to between 30-100° C., e.g., to between 30-80° C., e.g., to between 30-70° C., e.g., to about 40° C.), optionally with stirring.
6.12) Any of Process 6.8-6.11, wherein the mixture is cooled, e.g., cooled to <15° C., e.g., to <10° C., e.g., to between 4-8° C.
6.13) Any of Process 6 et seq., wherein the process comprises isolating purified 3α,7α,11β-trihydroxy-6α-ethyl-5β-cholan-24-oic acid.
6.14) Process 6.13, wherein 3α,7α,11β-trihydroxy-6α-ethyl-5β-cholan-24-oic acid is isolated by centrifugation and/or filtration.
6.15) Any of Process 6 et seq., wherein the purified 3α,7α,11β-trihydroxy-6α-ethyl-5β-cholan-24-oic acid is amorphous. 6.16) Any of Process 6 et seq., wherein the process further comprises formulating the purified 3α,7α,11-trihydroxy-6α-ethyl-5β-cholan-24-oic acid, e.g., by mixing the purified 3α,7α,11β-trihydroxy-6α-ethyl-5β-cholan-24-oic acid with a pharmaceutically acceptable excipient.

Further provided is a method (Method 1) of treating a disease or condition in a subject in need thereof, wherein the method comprises administering to the subject an effective amount of (or a pharmaceutical composition comprising) a crystalline form of 3α,7α,11β-trihydroxy-6α-ethyl-5β-cholan-24-oic acid, e.g., any of Crystalline Form 1 et seq., Crystalline Form 2 et seq., Crystalline Form 3 et seq., and Crystalline Form 4 et seq.
1.1) Method 1, wherein the disease or condition is FXR-mediated.
1.2) Method 1 or 1.1, wherein the disease or condition is selected from a cardiovascular disease, a liver disease, a lipid disorder, a gastrointestinal disease, a renal disease, a metabolic disease, cancer, a neurological disease, and an eye disease.
1.3) Method 1 or 1.1, wherein the disease or condition is a liver disease or condition.
1.4) Method 1.3, wherein the liver disease or condition is selected from the group consisting of primary biliary cholangitis (PBC), cerebrotendinous xanthomatosis (CTX), primary sclerosing cholangitis (PSC), drug induced cholestasis, intrahepatic cholestasis of pregnancy, parenteral nutrition associated cholestasis (PNAC), bacterial overgrowth or sepsis associated cholestasis, autoimmune hepatitis, viral hepatitis, alcoholic liver disease, acute decompensated liver disease (also known as acute-on-chronic liver failure), alcoholic hepatitis, severe alcoholic hepatitis (sAH), chronic vial hepatitis, nonalcoholic fatty liver disease (NAFLD), nonalcoholic steatohepatitis (NASH), liver transplant associated graft versus host disease, living donor transplant liver regeneration, congenital hepatic fibrosis, choledocholithiasis, granulomatous liver disease, intra- or extrahepatic malignancy, Sjogren's syndrome, Sarcoidosis, Wilson's disease, Gaucher's disease, hemochromatosis, and alpha 1-antitrypsin deficiency.
1.5) Any of Methods 1 or 1.1-1.4, wherein the disease or condition is as discussed in U.S. U.S. Pat. No. 9,611,289 or U.S. Pat. No. 11,066,437, U.S. Publications Nos. 2018/0256600 or 2020/0164005, or International Publication No. WO 2018/226724, Further provided is:
(i) a crystalline form of Compound 1 as described herein, e.g., any of Crystalline Form 1 et seq., Crystalline Form 2 et seq., Crystalline Form 3 et seq., and Crystalline Form 4 et seq., for use in any method or in the treatment of any disease or condition as described herein,
(ii) a crystalline form of 3α,7α,11β-trihydroxy-6α-ethyl-5β-cholan-24-oic acid (Compound 1) as described herein, e.g., any of Crystalline Form 1 et seq., Crystalline Form 2 et seq., Crystalline Form 3 et seq., and Crystalline Form 4 et seq., (in the manufacture of a medicament) for treating any disease or condition as described herein,
(iii) a pharmaceutical composition comprising a crystalline form of 3α,7α,11β-trihydroxy-6α-ethyl-5β-cholan-24-oic acid (Compound 1) as described herein, e.g., any of Crystalline Form 1 et seq., Crystalline Form 2 et seq., Crystalline Form 3 et seq., and Crystalline Form 4 et seq., in combination or association with a pharmaceutically acceptable diluent or carrier, e.g., any of Composition 1 et seq., and (iv) a pharmaceutical composition comprising a crystalline form of 3α,7α,11β-trihydroxy-6α-ethyl-5β-cholan-24-oic acid (Compound 1) as described herein, e.g., any of Crystalline Form 1 et seq., Crystalline Form 2 et seq., Crystalline Form 3 et seq., and Crystalline Form 4 et seq., in combination or association with a pharmaceutically acceptable diluent or carrier, e.g., any of Composition 1 et seq. for use in the treatment of any disease or condition as described herein.

The crystallinity, morphology, and properties of the crystals described herein, e.g., any of Crystalline Form 1 et seq., Crystalline Form 2 et seq., Crystalline Form 3 et seq., and Crystalline Form 4 et seq., may be determined by a number of methods, including, but not limited to single crystal X-ray diffraction, X-ray powder diffraction, polarizing optical microscopy, thermal microscopy, differential scanning calorimetry (DSC), thermogravimetric analysis (TGA), dynamic (water) vapor sorption, infared absorption spectroscopy, and Raman spectroscopy.

It is to be understood that an XRPD pattern of a given sample may vary (standard deviation) depending on the instrument used, the time and temperature of the sample when measured, and standard experimental errors. Therefore, the 2-theta values (°2θ), d-spacing values, heights, and relative intensity of the peaks will have an acceptable level of deviation. For example, the values may have an acceptable deviation of e.g., 20%, 15%, 10%, 5%, 3%, 2%, or 1%. In a particular embodiment, the 2-theta (°) values or the d-spacing (Å) values of the XRPD patterns of the crystalline forms described herein may have an acceptable deviation of ±0.2° and/or ±0.2 Å. Further, the XRPD patterns of the crystalline forms described herein may be identified by characteristic peak(s) as recognized by one skilled in the art. For example, the crystalline forms disclosed herein, e.g., any of Crystalline Form 1 et seq., 2 et seq., 3 et seq., or 4 et seq., may be identified by, e.g., two characteristic peaks, in some instances, three characteristic peaks, in another instance, five characteristic peaks. Therefore, the term “consistent with” shown in a particular figure refers to any crystal which has an XRPD having the major and/or characteristic and/or representative peaks as recognized by one skilled in the art.

It is also to be understood that the differential scanning calorimetry and thermogravimetric analysis thermograms of a given sample may vary (standard deviation) depending on the instrument used, the time and temperature of the sample when measured, and standard experimental errors. The temperature value itself may deviate by +10° C., preferably ±5° C., preferably ±3° C. of the reference temperature.

If multiple diffraction patterns are available, then assessments of particle statistics (PS) and/or preferred orientation (PO) are possible. If the effects of both PS and PO are determined to be negligible, then the XRPD pattern is representative of the powder average intensity for the sample and prominent peaks may be identified as “representative peaks.” “Characteristic peaks,” to the extent they exist, are a subset of representative peaks and are used to differentiate one crystalline polymorph from another crystalline polymorph (polymorphs being crystalline forms having the same chemical composition). Characteristic peaks are determined by evaluating which representative peaks, if any, are present in one crystalline polymorph of a compound against all other known crystalline polymorphs of that compound to within ±0.2 °2θ. Not all crystalline polymorphs of a compound necessarily have at least one characteristic peak.

As used herein, “XRPD” means X-ray powder diffraction.

As used herein, the term “solvate” refers to a crystal containing either stoichiometric or nonstoichiometric amounts of a solvent incorporated within the crystal structure.

As used herein, the term “amorphous” refers to solids of disordered arrangements of molecules and do not possess a distinguishable crystal lattice.

As used herein, “soluble” means the dissolution of 3α,7α,11β-trihydroxy-6αethyl-5β-cholan-24-oic acid is >10 mg/ml, e.g., >200 mg/ml.

As used herein, “poorly soluble” means the dissolution of 3α,7α,11β-trihydroxy-6α-ethyl-5β-cholan-24-oic acid is <10 mg/ml.

The International Council for Harmonisation of Technical Requirements for Pharmaceuticals for Human Use (ICH) provides guidance as to what amounts of residual solvents may be considered safe in pharmaceuticals. The guidance can be revised as more data becomes available. Class 2 solvents can be used but are “solvents to be limited.” Class 3 solvents are “solvents with low toxic potential.” Examples of Class 3 solvents are tert-butyl methyl ether and heptane. The ICH Guideline for Residual Solvents may be found on the ICH website at www.ich.org.

As used herein, “solvent” means a substance (e.g., a liquid) that can be used in pharmaceutical manufacturing, for instance, ICH class 2 and 3 solvents.

The terms “pharmaceutical” or “pharmaceutically acceptable” when used herein as an adjective, means substantially non-toxic and substantially non-deleterious to the recipient.

By “pharmaceutical formulation” it is further meant that the carrier, solvent, and/or excipient must be compatible with Compound 1. It is understood by those of ordinary skill in this art that the terms “pharmaceutical formulation” and “pharmaceutical composition” are generally interchangeable, and they are so used for the purposes of this application.

As used herein, the terms “treat,” “treating,” or “treatment” mean decreasing the symptoms, markers, and/or any negative effects of a condition in any appreciable degree in a subject who currently has the condition. In some embodiments, treatment may be administered to a subject who exhibits only early signs of the condition for the purpose of decreasing the risk of developing the disease or condition.

As used herein, “subject” means a human or animal (in the case of an animal, more typically a mammal). In one aspect, the subject is a human. Such subject can be considered to be in need of treatment with an FXR agonist.

As used herein, farnesoid X receptor or FXR refers to all mammalian forms of such receptor including, for example, alternative splice isoforms and naturally occurring isoforms (see, e.g., Huber et al., Gene 290:35-43 (2002)). Representative FXR species include, without limitation rat FXR (Gen Bank Accession No. NM_021745) mouse FXR (Genbank Accession No. NM_009108), and human FXR (GenBank Accession No. NM_005123).

3α,7α,11β-trihydroxy-6α-ethyl-5β-cholan-24-oic acid (Compound 1) may be synthesized as described in U.S. Pat. Nos. 9,611,289 and 11,066,473 and Pellicciari, R. et al., Journal of Medicinal Chemistry, 2016, 59, 9201-9214.

### Examples

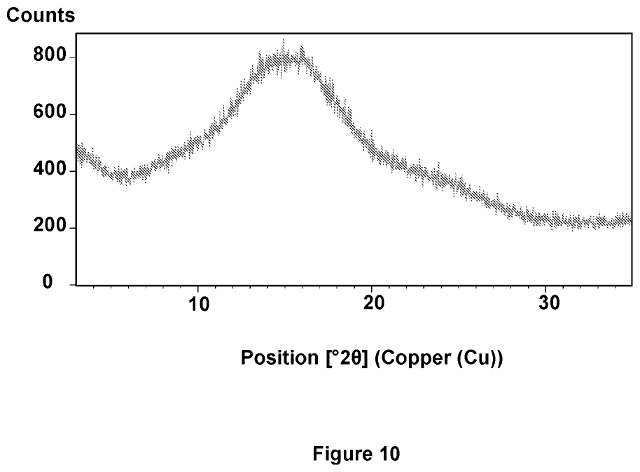

Starting material for Examples 2-8 is amorphous and non-solvated with a purity of 96.2% w/w by HPLC-CAD. An XRPD of the starting material is in FIG. 10. The starting material for Example 9 is the product from Example 3. The starting material for Example 10 is amorphous and non-solvated with a purity of 93.2% by CAD-HPLC.

### Abbreviations

h=hour(s)
tBME=tert-butyl methyl ether
RT=room temperature

### Example 1—General Procedures

### Example 1A—X-ray Powder Diffraction (XRPD)

XRPD analysis is carried out on a PANalytical X′pert pro with PIXcel detector (128 channels), scanning the samples between 3 and 35° 2θ. The material is gently ground to release any agglomerates and loaded onto a multi-well plate with Mylar polymer film to support the sample. The multi-well plate is then placed into the diffractometer and analyzed using Cu K radiation (α₁ λ=1.54060 Å; α₂=1.54443 Å; β=1.39225 Å; α₁: α₂ ratio=0.5) running in transmission mode (step size 0.0130° 20, step time 18.87s) using 40 kV/40 mA generator settings. Data are visualized and images generated using the HighScore Plus 4.7 desktop application (PANalytical, 2017).

### Example 1B—Thermogravimetric Analysis (TGA)

Approximately 5 mg of material is weighed into an open aluminum pan and loaded into a simultaneous thermogravimetric/differential thermal analyzer (TG/DTA) and held at room temperature. The sample is then heated at a rate of 10° C./min from 20° C. to 300 or 400° C. during which time the change in sample weight is recorded along with any differential thermal events (DTA). Nitrogen is used as the purge gas, at a flow rate of 300 cm³/min.

### Example 1C—Differential Scanning Calorimetry (DSC)

Approximately 5 mg of material is weighed into an aluminum DSC pan and sealed non-hermetically with a pierced aluminum lid. The sample pan is then loaded into a Seiko DSC6200 (equipped with a cooler) cooled and held at 20° C. Once a stable heat-flow response is obtained, the sample and reference are heated to 220° C. at scan rate of 10° C./min and the resulting heat flow response monitored. Nitrogen is used as the purge gas, at a flow rate of 50 cm³/min.

### Example 1D—Karl Fischer Coulometric Titration (KF)

Approximately 15-20 mg of solid material is accurately weighed into a vial. The solid is then manually introduced into the titration cell of a Mettler Toledo C30 Compact Titrator.

The vial is back-weighed after addition of the solid and the weight of the added solid entered on the instrument. Titration is initiated once the sample had fully dissolved in the cell. The water content is calculated automatically by the instrument as a percentage and the data printed. Two titrations are carried out for each solid, with the results reported as an average of the two.

### Example 1E—Dynamic Vapour Sorption (DVS)

Approximately 10-20 mg of sample is placed into a mesh vapour sorption balance pan and loaded into a DVS Advantage dynamic vapour sorption balance by Surface Measurement Systems. The sample is subjected to a ramping profile from 40-90% relative humidity (RH) at 10% increments, maintaining the sample at each step until a stable weight has been achieved (dm/dt 0.004%, minimum step length 30 minutes, maximum step length 500 minutes) at 25° C. After completion of the sorption cycle, the sample is dried using the same procedure to 0% RH and then a second sorption cycle back to 40% RH. Two cycles are performed. The weight change during the sorption/desorption cycles are plotted, allowing for the hygroscopic nature of the sample to be determined. XRPD analysis is then carried out on any solid retained.

### Example 2—Crystalline Form 1

100 mg of amorphous Compound 1 is dissolved in 10 mL of tBME:hexane 80:20 v/v. The solution is uncapped and allowed to evaporate at ambient temperature (ca. 20° C.). The XRPD of the collected material shows it to be amorphous. The material is re-suspended in 700 μL of tBME:hexane 80:20 v/v with magnetic stirring. Precipitate forms. A sub-sample is removed and analyzed by XRPD. The material is dried at ambient temperature under vacuum for ca. 4.5 h. XRPD analysis is carried out. The XRPD is consistent with FIG. 1. The material has a purity of 97.2% w/w.

### Example 3—Crystalline Form 1

325 mg of amorphous Compound 1 is suspended in 2 mL tBME:heptane 80:20 v/v. An additional 2 mL tBME:heptane 80:20 v/v is added to make the slurry more mobile. The mixture is stirred at ambient temperature for 10 min. A subsample of the slurry is removed and analysed by XRPD. The XRPD is consistent with FIG. 1. The slurry is filtered using Buchner filtration under vacuum. Recovered solids are dried under vacuum at ambient temperature for ca. 16-17 h.

### Example 4—Crystalline Form 1

30 mg of amorphous Compound 1 is suspended in tBME and stirred at 25° C. for 15 minutes. Anti-solvent addition is carried out in 100 μL aliquots, which are added dropwise where required. All mixtures are stirred for a further ca. 18 h at 25° C. Solids are isolated by centrifugation where possible and analyzed by XRPD. The XRPDs are consistent with Form 1. Solids are dried for 1 h at ambient temperature under vacuum.

TABLE 1

| | Solvent | | | | | HPLC-CAD |
|---|---|---|---|---|---|---|
| | Volume | Anti- | Anti-Solvent | Obervations | | Purity (% |
| Solvent | (μL) | Solvent | Volume (μL) | (18 h) | XRPD¹ | w/w) |
| tBME | 150 | N/A | N/A | Slurry | 1 | N/A |
| tBME | 150 | Heptane | 700 | Slurry | 1 | 90.0 |
| tBME | 500 | N/A | N/A | Slurry | 1 | 96.7 |

¹1 = Form 1

In all experiments, dissolution of the input material is observed followed by precipitation of solids (precipitation occurs before heptane addition). Purity of Form 1 is highest where tBME is the solvent. ¹H NMR analysis of Form 1 from 500 μL tBME shows no evidence of degradation and 0.67 equivalents tBME.

### Example 5—Crystalline Form 1

30 mg amorphous Compound 1 is suspended in 1 mL of tBME-saturated water (10 mL water and 2 mL tBME shaken vigorously for 5 min at ambient temperature, water layer removed) in duplicate. Mixtures are stirred at 25° C. for 1 h. 100 μL of tBME is added to the second mixture. Stirred at 25° C. for ca. 18 h. Where solids are observed, the material is analyzed by XRPD.

TABLE 2

| | Solvent | Additional | Observations | |
|---|---|---|---|---|
| Solvent System | Volume (μL) | tBME (μL) | (18 h) | XRPD¹ |
| tBME-Saturated | 1000 | N/A | Solids | 1 |
| Water | | | | |
| tBME-Saturated | 1000 | 100 | Two | N/A |
| Water | | | immiscible | |
| | | | layers | |

¹1 = Form 1

These experiments indicate that Crystalline Form 1 forms even where concentration of tBME is low.

### Example 6—Crystalline Form 1

500 mg of amorphous Compound 1 is suspended in 6 mL of tBME:heptane 80:20 v/v. Mixture is stirred at ambient temperature for ca. 3.5 h. Slurry is filtered using Buchner filtration under vacuum. Wet solids are analyzed by XRPD. Solids are dried under vacuum at ambient temperature for ca. 1 h. Crystalline Form 1 is prepared with a purity of 97.7% by HPLC-CAD. An XRPD pattern of the product is shown in FIG. 1.

¹H NMR shows 0.7 equivalents of tBME. TG analysis shows a loss of 12.7 wt. % (0.7 equivalents tBME) from the onset of heating to ca. 120° C. DT analysis shows an endothermic event associated with de-solvation with onset 104° C. (peak at 111° C.). DSC analysis shows an endothermic event with onset 104° C. (peak at 110° C.) in the first heating cycle. There are no significant events in the cool or second heat cycle. An overlay of a thermogravimetric analysis (TGA) thermogram and a differential analysis thermogram of the product is shown in FIG. 2. A differential scanning calorimetry thermogram of the product is shown in FIG. 3. KF analysis shows a moisture content of 0.6% w/w.

A dynamic vapour sorption analysis of Crystalline Form 1 is in FIG. 4. DVS analysis of Crystalline Form 1 shows an initial mass loss on the first sorption cycle before a slight uptake is observed. There is a loss of 1.9 wt. % on the first desorption cycle. There is an uptake of 1.1 wt. % (0.3 equivalents water) on the second sorption between 0 and 90% RH, followed by a loss of about 1.25 wt. % on desportion. There is a loss of 1.2 wt. % difference from the input to the end of the experiment. There is a slight loss of crystallinity post-DVS with some broadening of peaks which may be due to loss of tBME during the desorption cycles. Crystalline Form 1 remains as the bulk form.

Results of 7 day stability studies on Crystalline Form 1 are in Table 3.

TABLE 3

| Condition | XRPD¹ | HPLC-CAD Purity (% w/w) |
|---|---|---|
| Ambient Light | 1 | 97.5 |
| 40° C./75% RH | 1² | 97.1 |
| 80° C. | 1² | 96.4 |

¹1 = Form 1
²Loss in crystallinity

### Example 7-Crystalline Forms 1, 2, 3, and 4

30 mg of amorphous Compound 1 or Form 1 of Compound 1 (which may be prepared as laid out in Examples above) is suspended in solvent/solvent mixture and stirred at 50° C. Mixture is stirred for 1 h and then further solvent or solid is added if necessary. Mixture is stirred for further ca. 20 h at 50° C. Slurries are isolated by centrifugation. XRPD analysis is carried out on solids. Solids are dried for 1 h at ambient temperature under vacuum. XRPD analysis is repeated on the dried solids.

TABLE 4

| | | Total | | | | HPLC- |
|---|---|---|---|---|---|---|
| | | Solvent | | | | CAD |
| | | Volume | Observations | XRPD | XRPD | Purity |
| Input¹ | Solvent System | (μL) | (21 h) | Wet¹ | Dry¹ | (% w/w) |
| A | Diisopropyl | 400 | Slurry | A | | |
| | Ether:Heptane | | | | | |
| | 80:20 v/v | | | | | |
| A | tBME:Hexane | 800 | Slurry + Crust | 1 | | |
| | 70:30 v/v | | | | | |
| A | tBME:Heptane | 800 | Slurry + Crust | 1 | | |
| | 70:30 v/v | | | | | |
| A | Toluene:tBME | 800 | Slurry + Crust | 3 (XRPD | A | 96.3 |
| | 80:20 v/v | | | consistent | | |
| | | | | with | | |
| | | | | FIG. 7) | | |
| A | Toluene:Ethanol | 200 | Clear Solution | N/A | | |
| | 50:50 v/v² | | | | | |
| A | Toluene | 1000 | Gel | A | | |
| A | Heptane | 800 | Slurry | A | | |
| A | Hexane | 1000 | Slurry | A | | |
| 1 | Diisopropyl | 400 | Slurry | 4 (XRPD | 4 (XRPD | 92.3 |
| | Ether:Heptane | | | consistent | consistent | |
| | 80:20 v/v | | | with | with FIG. | |
| | | | | FIG. 8) | 8, TGA/DTA | |
| | | | | | in FIG. 9) | |
| 1 | tBME:Hexane | 800 | Slurry + Crust | 1 | | |
| | 70:30 v/v | | | | | |
| 1 | tBME:Heptane | 800 | Slurry | 1 | | |
| | 70:30 v/v | | | | | |
| 1 | Toluene:tBME | 1000 | Mobile Gel- | 3 (XRPD | | |
| | 80:20 v/v | | like Solids | consistent | | |
| | | | | with | | |
| | | | | FIG. 7) | | |
| 1 | Toluene:Ethanol | 200 | Clear Solution | N/A | | |
| | 50:50 v/v | | | | | |
| 1 | Toluene | 1000 | Slurry | 2 (XRPD | A | 93.4 |
| | | | | consistent | | |
| | | | | with | | |
| | | | | FIG. 5) | | |
| 1 | Heptane | 1000 | Slurry | A | | |

¹A = amorphous; 1 = Form 1; 2 = Form 2; 3 = Form 3; 4 = Form 4
²30 mg additional solid is added to Toluene:Ethanol 50:50 v/v mixture.

Analysis of dried Form 3 (amorphous) from toluene:tBME 80:20 v/v by ¹H NMR spectroscopic analysis shows 0.3 equivalents of toluene with no tBME present.

Analysis of dried Form 4 from diisopropyl ether:heptane 80:20 v/v by ¹H NMR spectroscopic analysis shows 0.3 equivalents of diisopropyl ether with no heptane present. TG/DT analysis shows a loss of 7.5 wt. % from the onset of heating to ca. 160° C. (0.35 equivalents diisopropyl ether).

### Example 8—Crystalline Forms 1, 2, and 3

30 mg of amorphous Compound 1 is placed into a 1.5 mL vial. 50 μL aliquots of the solvent system is added to form a mobile slurry. Experiments are stirred at 40° C. for 30 min.

Additional solvent is added if necessary. Mixture is thermally cycled between 4° and 5° C. with 0.1° C./min cooling and heating rates and 1 h holds at 40 and 5° C. After 21 h cycling, additional solvent is added if thick slurry is observed and anti-solvent (heptane) is added if clear solution is observed. After a further 26 h thermal cycling, observations are recorded at 5° C. Material is isolated by centrifugation if necessary and analyzed by XRPD. XRPD plate is dried at ambient temperature under vacuum for 2 h. XRPD analysis is repeated.

TABLE 5

| | | | Observations | | |
|---|---|---|---|---|---|
| | Volume | Anti- | Post-Thermal | XRPD | XRPD |
| Solvent System | (μL) | Solvent | Cycling | Wet¹ | Dry¹ |
| tBME:Hexane | 250 | N/A | Slurry | 1 | 1 |
| 90:10 v/v | | | | | |
| tBME:Hexane | 250 | N/A | Slurry | 1 | 1 |
| 80:20 v/v | | | | | |
| tBME:Hexane | 150 | N/A | Slurry | 1 | 1 |
| 70:30 v/v | | | | | |
| tBME:Heptane | 250 | N/A | Slurry | 1 | 1 |
| 90:10 v/v | | | | | |
| tBME:Heptane | 250 | N/A | Slurry | 1 | 1 |
| 80:20 v/v | | | | | |
| tBME:Heptane | 200 | N/A | Slurry | 1 | 1 |
| 70:30 v/v | | | | | |
| Diisopropyl | 250 | N/A | Solids + Crust | A | A |
| Ether:Hexane | | | | | |
| 80:20 v/v | | | | | |
| Diisopropyl | 250 | N/A | Solids + Crust | 2 | 3 |
| Ether:Heptane | | | | | |
| 80:20 v/v | | | | | |
| 1-Butanol:Heptane | 50 | Heptane | Gum | A | A |
| 80:20 v/v | | (150 μL) | | | |
| Acetone:Heptane | 50 | Heptane | Gum | A | A |
| 80:20 v/v | | (150 μL) | | | |
| Ethanol:Heptane | 50 | Heptane | Oil | N/A | N/A |
| 80:20 v/v | | (150 μL) | | | |
| Ethyl | 50 | Heptane | Gum | A | A |
| Acetate:Heptane | | (150 μL) | | | |
| 80:20 v/v | | | | | |
| Methyl Ethyl | 50 | Heptane | Gum | A | A |
| Ketone:Heptane | | (150 μL) | | | |
| 80:20 v/v | | | | | |
| THF:Heptane | 50 | Heptane | Gum | A | A |
| 80:20 v/v | | (150 μL) | | | |
| Toluene:tBME | 650 | N/A | Gel | 2 (TGA/DTA | 3 |
| 80:20 v/v | | | | in FIG. 6) | |
| Toluene:Ethanol | 200 | N/A | Clear Solution | N/A | N/A |
| 80:20 v/v | | | | | |

¹A = amorphous; 1 = Form 1; 2 = Form 2; 3 = Form 3

Form 2 is recovered from diisopropyl ether:heptane 80:20 v/v and toluene:tBIVE 80:20 v/v. After drying, Form 2 converts to Form 3.

¹H NMR spectroscopic analysis of Form 2 shows 0.6 equivalents toluene. TG analysis identifies a total loss of 19.5 wt. % (1.1 equivalents toluene or 1.2 equivalents tBME) from the onset of heating to ca. 300° C. before degradation. DT analysis shows two small broad endothermic events, the first of which has an onset of 55° C. (peak at 71° C.) and the second with onset 126° C. (peak at 139° C.). The thermal events may be related to de-solvation and melt.

### Example 9—Crystalline Forms 1, 2, and 3

20 mg of Form 1 from Example 3 is weighed into 1.5 mL vials. 50 μL aliquots of the solvent system is added to form a mobile slurry at 40° C. with stirring. Mixture is thermally cycled between 4° and 5° C. with 0.1° C./min cooling and heating rates and 1 h holds at 40 and 5° C. After 68 h thermal cycling, slurries are isolated and analysed by XRPD. The XRPD plate is dried under vacuum at ambient temperature for 3.5 h and XRPD analysis is repeated. Anti-solvent (heptane for all, except toluene:ethanol where toluene is added) is added to all experiments which remain solutions. After a further 48 h thermal cycling, material is isolated by centrifugation. XRPD analysis is carried out. The XRPD plate is dried under vacuum at ambient temperature for 21 h and XRPD analysis is repeated.

TABLE 6

| | Volume | Anti- | Observations | XRPD | XRPD |
|---|---|---|---|---|---|
| Solvent System | (μL) | Solvent | on Isolation | Wet¹ | Dry¹ |
| Diisopropyl | 200 | N/A | Slurry | 1 + 3 | 1 + 3 |
| Ether:Hexane 50:50 v/v | | | | | |
| Diisopropyl | 200 | N/A | Slurry | 1 + 3 | 1 + 3 |
| Ether:Heptane 50:50 v/v | | | | | |
| 1-Butanol:Heptane | 50 | Heptane | Gum | A | A |
| 50:50 v/v | | (200 μL) | | | |
| Acetone:Heptane 50:50 v/v | 50 | Heptane | Gum | A | A |
| | | (200 μL) | | | |
| Ethanol:Heptane 50:50 v/v | 50 | Heptane | Oil | N/A | N/A |
| | | (200 μL) | | | |
| Ethyl Acetate:Heptane | 150 | Heptane | Gum | A | A |
| 50:50 v/v | | (200 μL) | | | |
| Methyl Ethyl | 150 | Heptane | Gum | 1 | A |
| Ketone:Heptane 50:50 v/v | | (150 μL) | | | |
| THF:Heptane 50:50 v/v | 50 | Heptane | Gum | A | A |
| | | (100 μL) | | | |
| Toluene:tBME 50:50 v/v | 150 | N/A | Slurry | 1 | 1 |
| Toluene:Ethanol 50:50 v/v | 50 | Toluene | Gel | | |
| | | (200 μL) | | 2 | A |

¹A = amorphous; 1 = Form 1; 2 = Form 2; 3 = Form 3

Partially crystalline Form 2 is recovered form toluene:ethanol with final ratio 10:90 v/v, after anti-solvent addition.

### Example 10—Scaled-up Crystallization

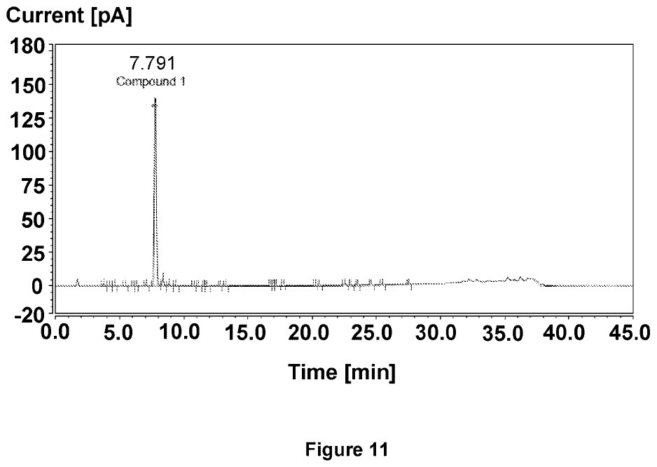

Starting material is amorphous Compound 1 with a purity by CAD-HPLC of 93.2%. Chromatogram of starting material is shown in FIG. 11.

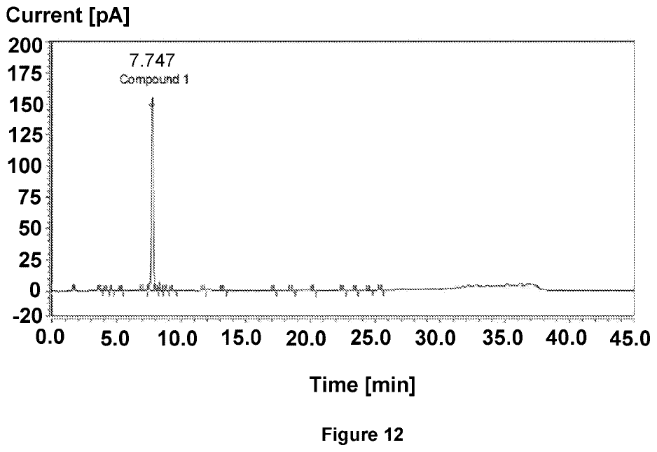

92.3 g of the starting material is co-evaporated three times with 600 mL of toluene in vacuum at 40° C. The residue is co-evaporated another 2 times with 300 mL of MtBE. The remaining solid is dissolved in 600 mL of MtBE and filtered through a glass fiber filter. The filtrate is evaporated to give a white, solid residue. The residue is suspended in 554 mL of MtBE/n-heptane 8:2 (443 mL+111 mL). The suspension is heated to 50° C. The suspension remains. The suspension is cooled to 22° C. within 3 hours. A crystalline slurry forms. The crystals are collected by filtration. The filter cake is washed with 2×200 mL of MtBE/n-heptane 1:1 v/v and dried in vacuum at 22° C. overnight. Yield: 97.3 g (86% yield). CAD-HPLC: 96.2 area % (see FIG. 12).

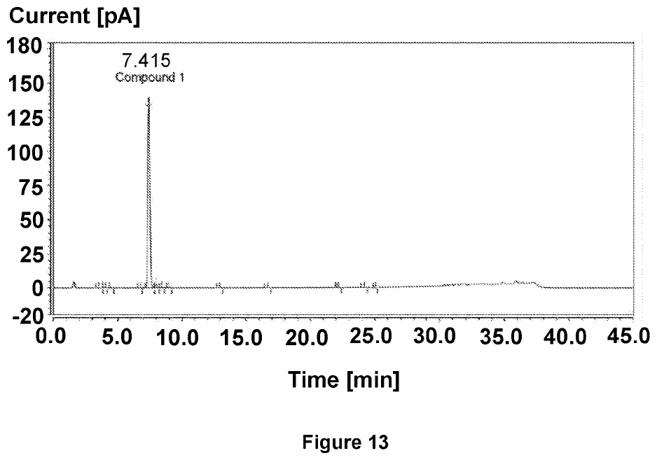

A 2L round bottom flask is charged with 94.7 g (180 mmol) of 3α,7,11β-trihydroxy-6α-ethyl-5β-cholan-24-oic acid crystals from above. 600 mL of 0.3 M NaOH solution (180 mmol) is added and the mixture is stirred for 30 minutes at 22° C. Solution is pH 9. 300 mL of tert-butyl methyl ether/water are removed by distillation at 50° C. under vacuum. The distillate is replaced by 300 mL of water and another 300 mL are evaporated under vacuum. The concentrate is acidified to pH 3.5 by addition of 80 mL of 1 M HCl+240 mL of 0.5 M HCl to provide a slurry. The slurry is warmed to 40° C. for 45 min. The mixture is cooled to RT and kept in at 4-8° C. overnight. The solid is filtered and the filter cake is washed 4×200 mL+1×360 mL of water. The product is dried in vacuum at 40° C. for 3 days. Yield: 78.2 g (84% yield). CAD-HPLC: 96.3 area % (see FIG. 13). Purity of 3α,7α,11β-trihydroxy-6α-ethyl-5β-cholan-24-oic acid is increased from 93 area % to 96 area %.

1. A tert-butyl methyl ether solvate of 3α,7α,11β-trihydroxy-6α-ethyl-5β-cholan-24-oic acid.

2. A crystalline form of 3α,7α,11β-trihydroxy-6α-ethyl-5β-cholan-24-oic acid, wherein the crystalline form exhibits at least one of an XRPD pattern comprising at least three of the following 2-theta (°) values: 5.4, 7.8, 10.9, 11.0, 12.4, 14.4, 15.5, 16.4, 16.7, 17.3, 18.3, and 19.7, wherein the XRPD is obtained using Cu K radiation and each of the 2-theta (°) values has an acceptable deviation of ±0.2°, or an XRPD pattern comprising at least three of the following d-spacing (Å) values: 16.2, 11.4, 8.1, 7.1, 6.1, 5.7, 5.4, 5.3, 5.1, 4.9, and 4.5.

3. The crystalline form of 3α,7α,11β-trihydroxy-6α-ethyl-5β-cholan-24-oic acid of claim 2, wherein the crystalline form exhibits at least one of an XRPD pattern comprising 2-theta (°) values of 5.4, 7.8, 10.9, 11.0, 12.4, 14.4, 15.5, 16.4, 16.7, 17.3, 18.3, and 19.7, wherein the XRPD is obtained using Cu K radiation and each of the 2-theta (°) values has an acceptable deviation of ±0.2°, or an XRPD pattern comprising d-spacing (Å) values of 16.2, 11.4, 8.1, 7.1, 6.1, 5.7, 5.4, 5.3, 5.1, 4.9, and 4.5.

4. The crystalline form of 3α,7α,11β-trihydroxy-6α-ethyl-5β-cholan-24-oic acid of claim 2, wherein the crystalline form exhibits at least one of an XRPD pattern comprising 2-theta (°) values of 5.4, 7.8, 9.4, 10.9, 11.0, 12.4, 14.4, 15.5, 16.4, 16.7, 17.3, 18.3, 19.2, 19.7, 20.0, 20.5, 22.0, 22.7, 23.0, 23.5, 24.3, 24.6, 25.0, 25.7, 26.4, 27.7, 28.6, 29.7, 30.3, 30.7, 31.2, 31.7, 32.7, and 34.1, wherein the XRPD is obtained using Cu K radiation and each of the 2-theta (°) values has an acceptable deviation of 0.2°, or an XRPD pattern comprising d-spacing (Å) values of 16.2, 11.4, 9.4, 8.1, 7.1, 6.1, 5.7, 5.4, 5.3, 5.1, 4.9, 4.6, 4.5, 4.4, 4.3, 4.0, 3.9, 3.8, 3.7, 3.6, 3.5, 3.4, 3.2, 3.1, 3.0, 2.9, 2.8, 2.7, and 2.6.

5. The crystalline form of 3α,7α,11β-trihydroxy-6α-ethyl-5β-cholan-24-oic acid of claim 2, wherein the crystalline form exhibits an XRPD pattern corresponding to FIG. 1, wherein the XRPD is obtained using Cu K radiation.

6. A crystalline form of 3α,7α,11β-trihydroxy-6α-ethyl-5β-cholan-24-oic acid, wherein the crystalline form exhibits at least one of:
an XRPD pattern comprising at least three of the following 2-theta (°) values: 4.4, 5.5, 6.0, 7.3, 8.5, 8.9, 9.5, 10.3, 10.7, 11.4, 12.2, 12.6, 14.1, 14.8, 16.5, 17.1, 17.6, 18.1, 18.7, 19.1, 19.3, 19.5, 20.0, 20.5, 20.9, 21.2, 21.9, 22.6, 23.3, 24.3, and 25.2, wherein the XRPD is obtained using Cu K radiation and each of the 2-theta (°) values has an acceptable deviation of ±0.2°; or
an XRPD pattern comprising at least three of the following d-spacing (Å) values: 19.9, 16.2, 14.7, 12.1, 10.4, 9.9, 9.4, 8.6, 8.2, 7.8, 7.2, 7.0, 6.3, 6.0, 5.4, 5.2, 5.0, 4.9, 4.8, 4.6, 4.5, 4.4, 4.3, 4.2, 4.1, 3.9, 3.8, 3.7, and 3.5; or
an XRPD pattern comprising at least three of the following 2-theta (°) values: 5.8, 8.5, 9.1, 9.8, 10.4, 13.0, 15.0, 16.1, 17.5, 18.1, and 22.9, wherein the XRPD is obtained using Cu K radiation and each of the 2-theta (°) values has an acceptable deviation of ±0.2°; or
an XRPD pattern comprising at least three of the following d-spacing (Å) values: 15.1, 10.4, 9.7, 9.0, 8.5, 6.8, 5.9, 5.5, 5.1, 4.9, and 3.9; or
an XRPD pattern comprising at least three of the following 2-theta (°) values: 5.8, 6.2, 7.4, 9.5, 11.7, 12.4, 14.2, 14.5, 15.1, 15.6, 16.0, 17.0, 17.4, 18.2, 19.0, and 20.8, wherein the XRPD is obtained using Cu K radiation and each of the 2-theta (°) values has an acceptable deviation of ±0.2°; or
an XRPD pattern comprising at least three of the following d-spacing (Å) values: 15.3, 14.2, 12.0, 9.3, 7.6, 7.1, 6.2, 6.1, 5.9, 5.7, 5.5, 5.2, 5.1, 4.9, 4.7, and 4.3.

7. The crystalline form of 3α,7α,11β-trihydroxy-6α-ethyl-5β-cholan-24-oic acid of claim 6, wherein the crystalline form exhibits at least one of:
an XRPD pattern comprising 2-theta (°) values of: 4.4, 5.5, 6.0, 7.3, 8.5, 8.9, 9.5, 10.3, 10.7, 11.4, 12.2, 12.6, 14.1, 14.8, 16.5, 17.1, 17.6, 18.1, 18.7, 19.1, 19.3, 19.5, 20.0, 20.5, 20.9, 21.2, 21.9, 22.6, 23.3, 24.3, and 25.2, wherein the XRPD is obtained using Cu K radiation and each of the 2-theta (°) values has an acceptable deviation of 0.2°; or
an XRPD pattern comprising d-spacing (Å) values of: 19.9, 16.2, 14.7, 12.1, 10.4, 9.9, 9.4, 8.6, 8.2, 7.8, 7.2, 7.0, 6.3, 6.0, 5.4, 5.2, 5.0, 4.9, 4.8, 4.6, 4.5, 4.4, 4.3, 4.2, 4.1, 3.9, 3.8, 3.7, and 3.5; or
an XRPD pattern comprising 2-theta (°) values of: 5.8, 8.5, 9.1, 9.8, 10.4, 13.0, 15.0, 16.1, 17.5, 18.1, and 22.9, wherein the XRPD is obtained using Cu K radiation and each of the 2-theta (°) values has an acceptable deviation of ±0.2°; or
an XRPD pattern comprising d-spacing (Å) values of: 15.1, 10.4, 9.7, 9.0, 8.5, 6.8, 5.9, 5.5, 5.1, 4.9, and 3.9; or
an XRPD pattern comprising 2-theta (°) values of: 5.8, 6.2, 7.4, 9.5, 11.7, 12.4, 14.2, 14.5, 15.1, 15.6, 16.0, 17.0, 17.4, 18.2, 19.0, and 20.8, wherein the XRPD is obtained using Cu K radiation and each of the 2-theta (°) values has an acceptable deviation of ±0.2°; or
an XRPD pattern comprising d-spacing (Å) values of: 15.3, 14.2, 12.0, 9.3, 7.6, 7.1, 6.2, 6.1, 5.9, 5.7, 5.5, 5.2, 5.1, 4.9, 4.7, and 4.3.

8. The crystalline form of 3α,7α,11β-trihydroxy-6α-ethyl-5β-cholan-24-oic acid of claim 6, wherein the crystalline form exhibits at least one of:
an XRPD pattern comprising 2-theta (°) values of: 4.4, 5.5, 6.0, 7.0, 7.3, 7.9, 8.5, 8.9, 9.5, 10.3, 10.7, 11.4, 11.7, 12.2, 12.6, 12.9, 13.7, 14.1, 14.8, 16.5, 17.1, 17.6, 18.1, 18.7, 19.1, 19.3, 19.5, 20.0, 20.5, 20.9, 21.2, 21.9, 22.6, 23.3, 24.3, 24.5, 25.2, 25.5, 25.9, and 29.1, wherein the XRPD is obtained using Cu K radiation and each of the 2-theta (°) values has an acceptable deviation of ±0.2°; or
an XRPD pattern comprising d-spacing (Å) values of: 19.9, 16.2, 14.7, 12.6, 12.1, 11.1, 10.4, 9.9, 9.4, 8.6, 8.2, 7.8, 7.6, 7.2, 7.0, 6.8, 6.4, 6.3, 6.0, 5.4, 5.2, 5.0, 4.9, 4.8, 4.6, 4.5, 4.4, 4.3, 4.2, 4.1, 3.9, 3.8, 3.7, 3.6, 3.5, 3.4, and 3.1; or
an XRPD pattern comprising 2-theta (°) values of: 4.1, 5.8, 8.5, 9.1, 9.8, 10.4, 12.1, 13.0, 14.2, 15.0, 16.1, 17.5, 18.1, 19.0, 22.9, 24.9, and 26.2, wherein the XRPD is obtained using Cu K radiation and each of the 2-theta (°) values has an acceptable deviation of ±0.2°; or
an XRPD pattern comprising d-spacing (Å) values of: 21.4, 15.1, 10.4, 9.7, 9.0, 8.5, 7.3, 6.8, 6.2, 5.9, 5.5, 5.1, 4.9, 4.7, 3.9, 3.6, and 3.4; or
an XRPD pattern comprising 2-theta (°) values of: 5.8, 6.2, 7.4, 9.5, 11.7, 12.4, 14.2, 14.5, 15.1, 15.6, 16.0, 17.0, 17.4, 18.2, 19.0, 20.8, 23.5, 24.3, and 25.5, wherein the XRPD is obtained using Cu K radiation and each of the 2-theta (°) values has an acceptable deviation of ±0.2°; or
an XRPD pattern comprising d-spacing (Å) values of: 15.3, 14.2, 12.0, 9.3, 7.6, 7.1, 6.2, 6.1, 5.9, 5.7, 5.5, 5.2, 5.1, 4.9, 4.7, 4.3, 3.8, 3.7, and 3.5.

9. The crystalline form of 3α,7α,11β-trihydroxy-6α-ethyl-5β-cholan-24-oic acid of claim 6, wherein the crystalline form exhibits an XRPD pattern corresponding to any one of FIG. 5, FIG. 7, or FIG. 8, wherein the XRPD is obtained using Cu K radiation.

10. A process for purifying 3α,7α,11 β-trihydroxy-6α-ethyl-5β-cholan-24-oic acid, wherein the process comprises mixing the crystalline form of 3α,7α,11β-trihydroxy-6α-ethyl-5β-cholan-24-oic acid of claim 2 with a solvent and isolating the purified 3α,7α,11β-trihydroxy-6α-ethyl-5β-cholan-24-oic acid.

11. The process of claim 10, wherein the solvent is water.

12. The process of claim 10, wherein the solvent is a base or comprises a base.

13. The process of claim 10, wherein the solvent is an aqueous solution comprising a base.

14. The process of claim 13, wherein the aqueous solution comprising a base is added to pH 9.

15. The process of claim 14, wherein the base has a pKb≤1.

16. The process of claim 14, wherein the base is an alkali hydroxide base.

17. The process of claim 16, wherein the base is one or more of lithium hydroxide, potassium hydroxide, and sodium hydroxide.

18. The process of claim 17, wherein the base is sodium hydroxide.

19. The process of claim 16, wherein the process comprises mixing the crystalline form with 1-1.5 equivalents of the base.

20. The process of claim 14, wherein the process comprises mixing 3α,7α,11β-trihydroxy-6α-ethyl-5β-cholan-24-oic acid with an acid.

21. The process of claim 20, wherein the process comprises mixing with 1-1.5 equivalents of the acid.

22. The process of claim 20, wherein the acid has a pKa<0.

23. The process of claim 20, wherein the acid is hydrochloric acid.

24. The process of claim 10, wherein the process comprises isolating purified 3α,7α,11β-trihydroxy-6α-ethyl-5β-cholan-24-oic acid.

25. The process of claim 24, wherein the purified 3α,7α,11β-trihydroxy-6α-ethyl-5β-cholan-24-oic acid is amorphous.
